(19) United States
(12) Patent Application Publication
(10) Pub. No.: US 2026/0144595 A1
(43) Pub. Date: May 28, 2026

(54) Systems and Methods for Anatomical and Device Optimization

(71) Applicant: DasiSimulations, LLC, Dublin, OH (US)

(72) Inventors: Taylor Nicole Sirset-Becker, Rancho Palos Verdes, CA (US); Lakshmi Prasad Dasi, Dublin, OH (US)

(21) Appl. No.: 19/403,140

(22) Filed: Nov. 27, 2025

Related U.S. Application Data
(60) Provisional application No. 63/725,609, filed on Nov. 27, 2024.

(51) Int. Cl. A61B 34/10; A61B 34/00

(52) U.S. Cl. CPC A61B 34/10; A61B 34/25; A61B 2034/104; A61B 2034/105; A61B 2034/108; A61B 2034/252

(57) ABSTRACT

A computer-implemented method includes obtaining data related to a heart medical procedure for a patient, the data including patient-specific data including 3D computational analysis generated from pre-procedural medical imaging, device data related to at least one device to be deployed in the heart medical procedure, and interactive input from medical personnel. The method includes generating, based on the data, options of modification strategies including device modifications to the at least one device and anatomical modifications to the patient's anatomy. The method includes simulating the heart medical procedure and generating a simulation outcome for each option of the modification strategies, selecting a modification strategy that corresponds to the simulation outcome having a desired heart medical procedure result, and generating procedural recommendations including device deployment instructions and modification parameters to guide the heart medical procedure in real life.

### CROSS-REFERENCE TO RELATED APPLICATIONS

The present application claims the benefit of priority under 35 U.S.C. § 119 from U.S. Provisional Patent Application No. 63/725,609 entitled “Systems and Methods for Anatomical and Device Optimization,” filed on Nov. 27, 2024, the disclosure of which is hereby incorporated by reference in its entirety for all purposes.

### TECHNICAL FIELD

The present specification generally relates to generative computational predictive models, and more specifically relates to generative computational predictive models and systems and methods for anatomical and device optimization for heart procedures.

### BACKGROUND

Structural heart diseases are increasingly treated with transcatheter implants such as transcatheter heart valves, closure devices etc. or surgical valve replacements such as mechanical, bioprosthetic or polymer based valves or other implants. However successful outcomes depend heavily on how well the prosthetic device interacts and conforms to the patient's unique anatomy as well as the anatomy conforming to the device (coupled interaction). In practice, many patients present with anatomical variations-such as irregular annular shapes, asymmetric or heavy calcification, small sinuses, low coronary ostia, or highly angulated valve roots-that differ from the idealized geometries assumed during valve design. These discrepancies can hinder proper expansion, sealing, and anchoring of the implant, and may impair leaflet motion or device positioning during deployment.

As a result, anatomical incompatibilities may lead to complications such as paravalvular leak, coronary obstruction, valve migration, left ventricular outflow track (LVOT) obstruction, thrombosis, device migration, embolization, conduction disturbances, and reduced long-term durability. Current imaging and procedural planning tools also have limitations in predicting how devices will interact with complex or atypical cardiac structures. Further, while there have been recent advances in computer-based modeling such as PrecisionTAVI™ available from DASI Simulations LLC or FEops HEARTguide™, these modeling tools have not yet been integrated into optimization of implant devices or the optimization of the patient's anatomy. They simply predict the interaction and any complication but do not guide the physicians into potential optimization of the implant or the patient anatomy for reducing the predicted risks. Therefore, there remains a need for improved valve devices, systems, and deployment techniques by way of providing guidance for optimization of the device and/or anatomy that can better accommodate anatomical variability, predicted risk from standard modeling, and achieve reliable performance across a broader range of patient anatomies.

The description provided in the background section should not be assumed to be prior art merely because it is mentioned in or associated with the background section. The background section may include information that describes one or more aspects of the subject technology.

### SUMMARY

A computer-implemented method includes obtaining data related to a heart medical procedure for a patient, the data comprising patient-specific data comprising 3D computational analysis generated from pre-procedural medical imaging, device data related to at least one device to be deployed in the heart medical procedure, and interactive input from medical personnel; generating, based on the data, options of modification strategies comprising device modifications to the at least one device and anatomical modifications to the patient's anatomy; simulating the heart medical procedure and generating a simulation outcome for each option of the modification strategies;

selecting a modification strategy that corresponds to the simulation outcome having a desired heart medical procedure result; and generating procedural recommendations comprising device deployment instructions and modification parameters to guide the heart medical procedure in real life.

A system includes a memory comprising instructions; and a processor configured to execute the instructions which, when executed, cause the processor to: obtain data related to a heart medical procedure for a patient, the data comprising patient-specific data comprising 3D computational analysis generated from pre-procedural medical imaging, device data related to at least one device to be deployed in the heart medical procedure, and interactive input from medical personnel; generate, based on the data, options of modification strategies comprising device modifications to the at least one device and anatomical modifications to the patient's anatomy; simulate the heart medical procedure and generate a simulation outcome for each option of the modification strategies; select a modification strategy that corresponds to the simulation outcome having a desired heart medical procedure result; and generate procedural recommendations comprising device deployment instructions and modification parameters to guide the heart medical procedure in real life.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an anatomy of a biscuspid aortic valve modeled using the computer system disclosed herein.

FIGS. 2A, 2B, and 2C each illustrate a possible location of cut modeled using the computer system disclosed herein.

FIG. 3 illustrates characterization of split on raphe under balloon-expandable TAVR in comparison to cases wherein no anatomy modification is performed, modeled using the computer system disclosed herein.

FIG. 4 illustrates characterization of split on raphe under self-expandable TAVR in comparison to cases wherein no anatomy modification is performed, modeled using the computer system disclosed herein.

FIG. 5 shows split on raphe comparison between split and no anatomy modification, based on stretch analysis, modeled using the computer system disclosed herein.

FIG. 6 shows split on raphe comparison between split and no anatomy modification for balloon-expandable TAVR and self-expandable TAVR, modeled using the computer system disclosed herein.

FIGS. 7-10 show the results for the split adjacent to the raphe (shown in FIG. 2B), modeled using the computer system disclosed herein.

FIGS. 11-14 show results for the two splits (shown in FIG. 2C), modeled using the computer system disclosed herein.

FIG. 15 illustrates a different bicuspid patient case where a physician is exploring the reduction of volume from no reduction all the way to 4 cubic centimeter (c.c.) reduction in volume at increments of 1 c.c., modeled using the computer system disclosed herein.

FIGS. 16-20 show another bicuspid patient case of a type 0 bicuspid and the physician is exploring cutting both the leaflets, modeled using the computer system disclosed herein.

FIG. 21 shows a model training process using the computer system disclosed herein.

FIG. 22 shows a prediction of how the aperture in the Medtronic Evolut FX+ valve can be optimized with respect to the coronary ostium, modeled using the computer system disclosed herein.

FIG. 23 shows an exemplary interactive output display of a predicted final deployment configuration of the transcatheter device in the anatomy, modeled using the computer system disclosed herein.

FIGS. 24-26 show another exemplary interactive output display showing the orientation of the valve and the anatomy, modeled using the computer system disclosed herein.

FIG. 27 shows an example of using the computer system disclosed herein for optimization of future intervention.

FIG. 28 shows an exemplary computer-implemented method using the computer system disclosed herein.

FIG. 29 shows another exemplary computer-implemented method using the computer system disclosed herein.

FIGS. 30A and 30B illustrate examples of the computer system disclosed herein for predictive heart valve and procedure simulation.

FIGS. 31A and 31B show exemplary anatomical model data.

FIGS. 32-35 show exemplary deformed analytical models.

FIG. 36 shows an example of physician driven method to modify device intraoperatively based on instructions generated by the computer system disclosed herein.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various ways, all without departing from the scope of the present disclosure. Accordingly, the drawings and description are to be regarded as illustrative in nature and not restrictive.

It should be understood that while the disclosed systems and methods described below are in the context of soft tissue repair treatment procedures for various cardiovascular diseases, the disclosed technology can be extended and is applicable for other fields such as orthopedics, plastic surgery, and other appropriate fields. The disclosed technology is applicable for any soft tissue repair or device implantation where a surgeon can manipulate the tissue or existing implants with or without devices (e.g. wires, cutting tools, plugs, electrosurgical wire, or similar manipulating and/or guiding devices etc.) and needs immediate prediction of functional outcomes of those repair or implantation steps on the organ/tissue being repaired or treated.

During structural heart procedures such as transcatheter heart valve implantation or left atrial appendage closure device implantation or other similar devices, the patient's anatomy is not entirely compatible with the device. The word “incompatible” is used loosely to convey “not optimal” or “not as designed for the device's original indication”. Examples of this incompatibility may be complex anatomy that creates either a size mismatch or presents a potential problem such as risk of coronary occlusion, risk of device leakage, risk of tissue rupture and bleeding, risk of device thrombosis etc. When these risks are predictable, it is possible to modify or customize the procedure by either modifying the anatomy or modifying the device using additional methods and techniques. One example of anatomical modification is the procedure called bioprosthetic or native aortic scallop intentional laceration to prevent iatrogenic coronary artery obstruction (BASILICA) technique, where an electrosurgical wire is introduced to lacerate or cut portions of the anatomy to create new flow paths that would otherwise be obstructed. Similar techniques in the mitral valve area include laceration of the anterior mitral leaflet to prevent outflow obstruction (LAMPOON) or septal correction to prevent iatrogenic left ventricular outflow tract obstruction (SCORPION) technique or Septal Ablation (with or without alcohol) or other similar techniques. Future devices such as the shortcut device (from PiCardia Inc.) can also replace the electro-surgical wire with the use of a sharp cutting element (e.g. a blade) on a transcatheter device. There may be other devices to resect tissue or failed leaflets using transcatheter capture techniques.

Examples of device modification include over-filling or under-filling the balloon that is used to expand the device thereby modifying the device from its original intended design, or use a transcatheter mechanism to turn a mechanical screw like element on the device to mechanically adjust the size Some delivery systems may calibrate the balloon volume to guide the physician to know the size of the device as a function of the inflation. Yet another example of device modification can be the angular alignment of the device to optimize access to adjacent anatomical structures such as the coronary ostia. In some heart valves it may be beneficial to orient the angular position to align certain cells in the stent frame (e.g., the geometric opening or the mesh-like pattern formed by the interconnected struts that make up the stent frame structure) to the coronary ostium or align the leaflet commissures to the native commissures (such as the aperture windows of the Evolut FX+device). There may be different competing interests for the optimal alignment. For example, it may be desirable to align to reduce the likelihood of future thrombosis (because of better blood flow patterns) or it may be desirable to align to maximize future access to the coronaries for future procedures such as coronary stenting after transcatheter valve replacement or a multitude of different factors that would motivate optimization steps.

While there are techniques available currently to be able to do these various anatomy or device modifications, the decision to perform an anatomy or device modification (or both) is not uniform. Physicians currently do not have a tool or model to help them craft a customization protocol for a given patient that will include all the different possible anatomy and device modification methods available and be able to help isolate the best possible anatomy and/or device modification for a given patient. Current computational models or digital twin models are only configured to output risk after implantation of the device per manufacturer's instructions for use (IFU), but these models are not capable of identifying the best possible set of anatomy/device modifications.

With the possibility to modify both the device and anatomy, it would be possible to enter a highly personalized approach to disease treatment that not only aims to prevent complications but also improve the performance and long-term durability of the intervention. The goal of this present invention disclosure is to introduce systems and simulation models that enable a portion (or entirety) of interventional or surgical procedure method be computer generated, including a prescribed step by step procedure or a portion of the procedure with choices for anatomy and device modifications with corresponding predicted outcomes, to achieve reduced risk for complication and maximize the likelihood for good hemodynamic and long term outcome at an individual patient specific level. These models can be a combination of data driven and physics driven models that can not only predict the structural and flow dynamic interactions between different modifying interventions but also have the capability to predict long-term durability, hemodynamics and remodeling of the patient's anatomy in order to be able to optimize for more long-term benefits for the patients. Once the model identifies the optimal set of modifications, it can generate the plan and help the physician execute these instructions during the procedure. Some of the implant devices or modification elements (e.g. cutting tool) could have real time feedback through sensors or visual feedback via intraoperative imaging modalities to further guide the modification real time during the procedure.

The present disclosure is about systems and methods to (a) predict the efficacy of different anatomy and/or device modifications so the decisions can be made whether anatomy/device modification is needed and what choices are available toward optimization and (b) predict modified method of interventional workflow to include the computational model to inform certain steps in the procedure including any real time generation of interventional steps if the procedure involves devices with feedback mechanisms built in.

The present disclosure is also about systems and methods including a computer-implemented procedural optimization platform for structural heart interventions. The system constructs patient-specific anatomical models from pre-procedural imaging, integrates device-specific mechanical properties and clinician input, and generates a set of candidate modification strategies comprising both anatomy-remodeling steps and implant-device deployment parameter tuning. A digital twin deformation engine performs predictive simulation-optionally in real time using reversible sensing feedback obtained during catheter-based navigation and expansion-to estimate final device geometry and orientation relative to critical anatomical landmarks including coronary ostia, annular root structures, and adjacent flow pathways. Simulation outcomes are algorithmically ranked using quantitative biomarkers such as paravalvular gaps, obstruction distances, and tissue stretch-based injury surrogates to identify an optimal modification protocol. The system outputs clinician-interactive procedural recommendations, including deployment orientation, inflation or expansion parameters, and leaflet-level or frame-level modification instructions, to reduce complication risk and improve immediate and long-term structural and hemodynamic outcomes. Unlike conventional planning tools limited to manufacturer's IFU risk reporting, the disclosed platform generates and ranks actionable anatomy and device modification strategies and accommodates diverse patient anatomies including bicuspid, angulated, or calcification-constrained morphologies.

### Example #1: Optimization for Bicuspid Aortic Valve Patients with Aortic Stenosis

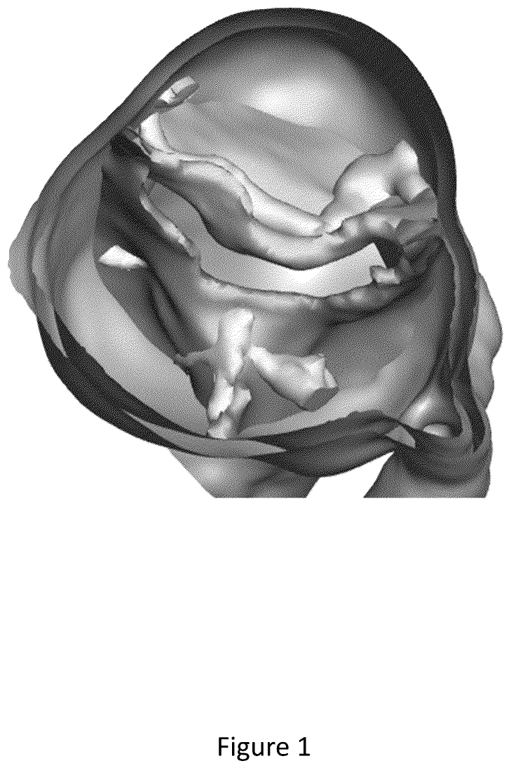

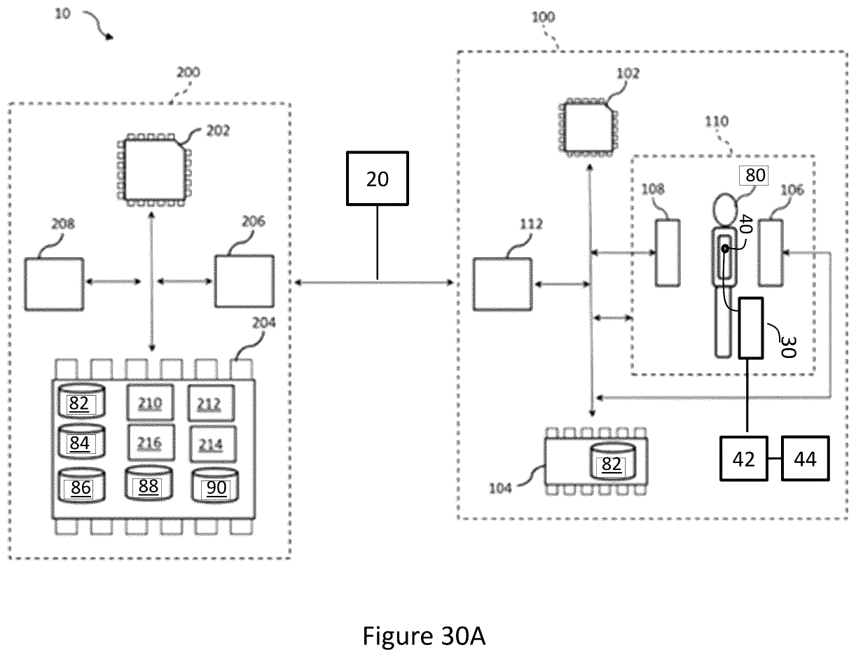

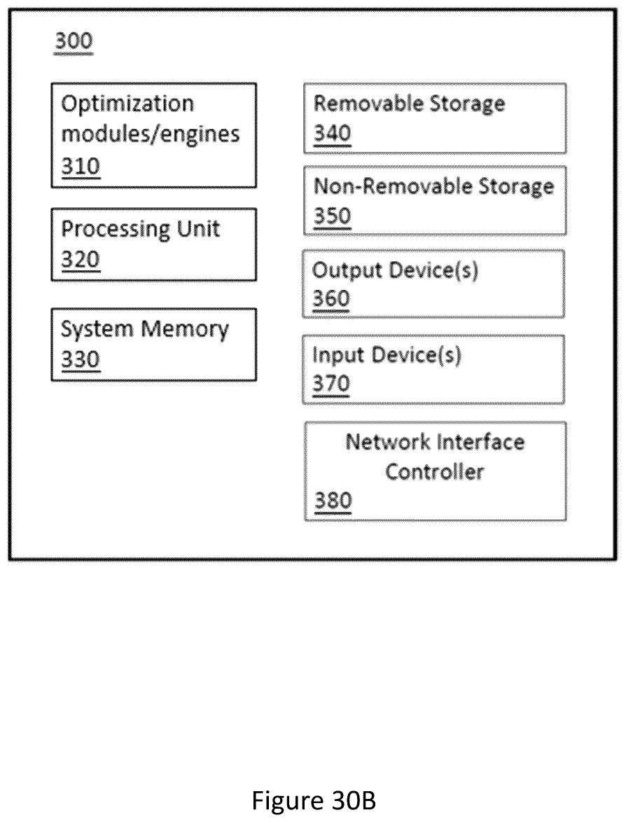

As an example of the present disclosure here we provide details of how the above systems and methods are implemented for transcatheter valve replacement in a bicuspid aortic stenosis patient. FIG. 1 shows a simulated anatomy of a bicuspid aortic valve of a patient based on the patient's CT scan subjected to segmentation methods. Physicians know that in this type of anatomy, the presence of the raphe is a challenge as it would inhibit the central expansion of the transcatheter aortic valve leading to potential long turn durability concerns or higher risk for thrombosis or hypoattenuated leaflet thickening (HALT). It would therefore be beneficial for the physician to understand the impact of introducing cuts (anatomical modification) but it is not clear where to introduce the cuts and how to judge which location to cut the leaflet. Therefore, this is an example where anatomy optimization may be helpful prior to deployment of a transcatheter valve to maximize the benefits of the treatment with longer durability with better device frame expansion. In this case, a computer system 10 (shown in FIGS. 30A and 30B) is used to decide and then guide the modification of the anatomy by introducing different types of lacerations or cuts to the leaflet during the procedure. The physician can interact with the computational system 10 and interactively place the cuts to the leaflet using an interactive graphical user interface 20, which may be a display or an interactive device such as an ipad or a computer browser with an app where the physician can use the computer mouse to position the cuts. If the cutting device has a feedback sensor or a method for the physician to gauge the progress of the cuts, the computer model can be configured to provide real time instructions and update the optimal guidance based on progress during the procedure monitored by various intraoperative and bio-signal monitors (flow, pressure etc.).

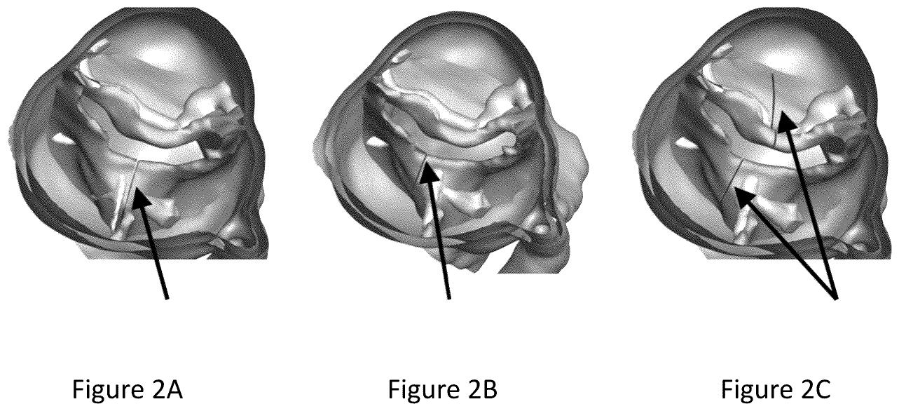

FIGS. 2A, 2B, and 2C show simulated possible locations of the cuts (indicated by arrows) simulated by the computer system 10. A physician can use the interactive device to modify the display of the respective cusps with the precise cuts where the physician would like to examine as possible modifications for the anatomy. The computer system 10 may implement the deletion of nodes and connectivity to model the lacerations as shown in FIGS. 2A, 2B, and 2C. The appropriate nodes may be selected using the interactive graphical user interface 20 to select and delete.

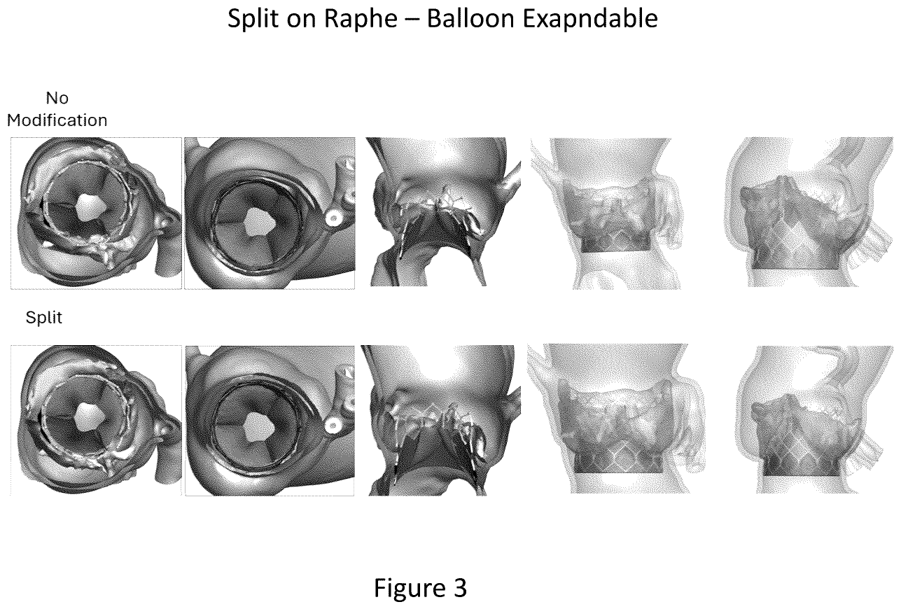

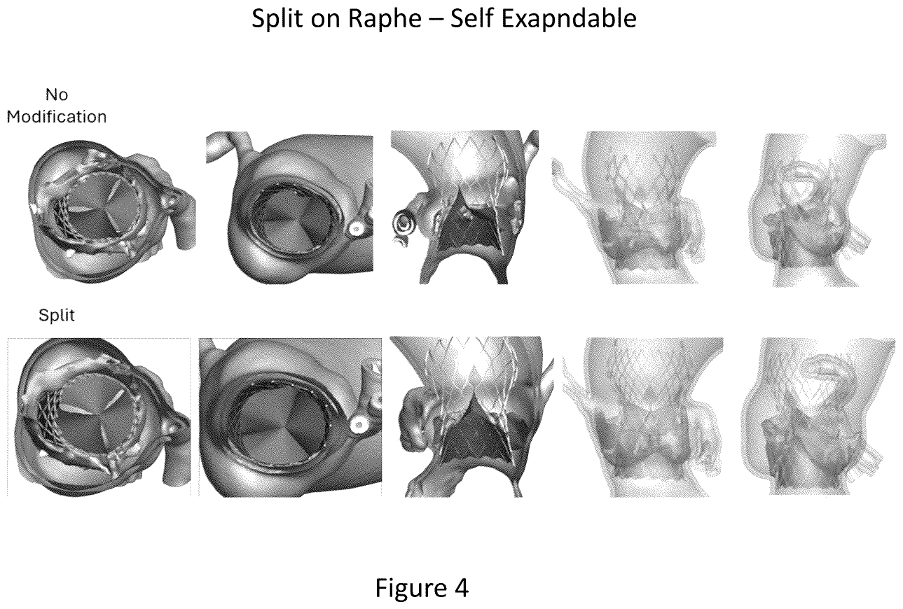

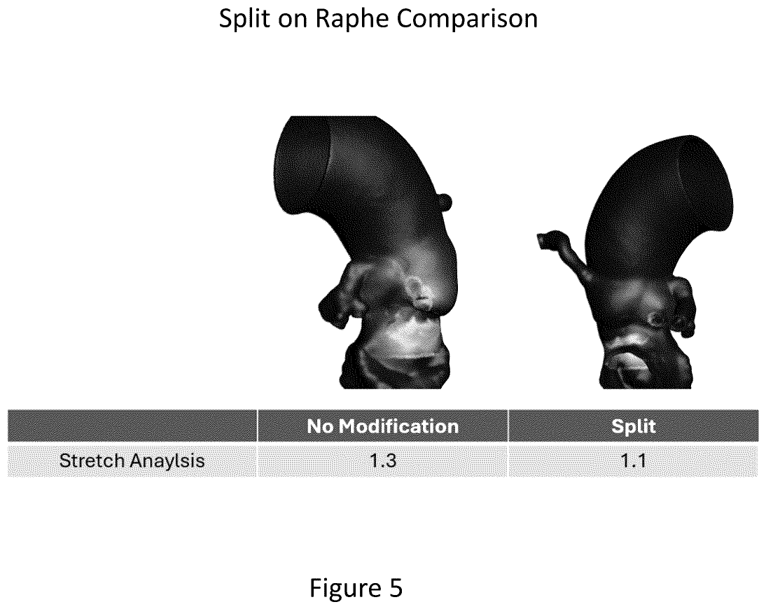

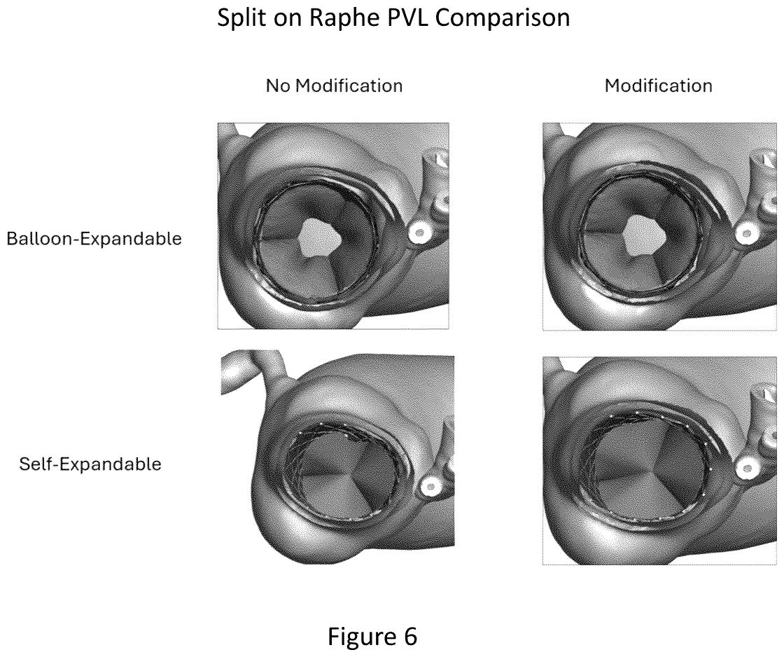

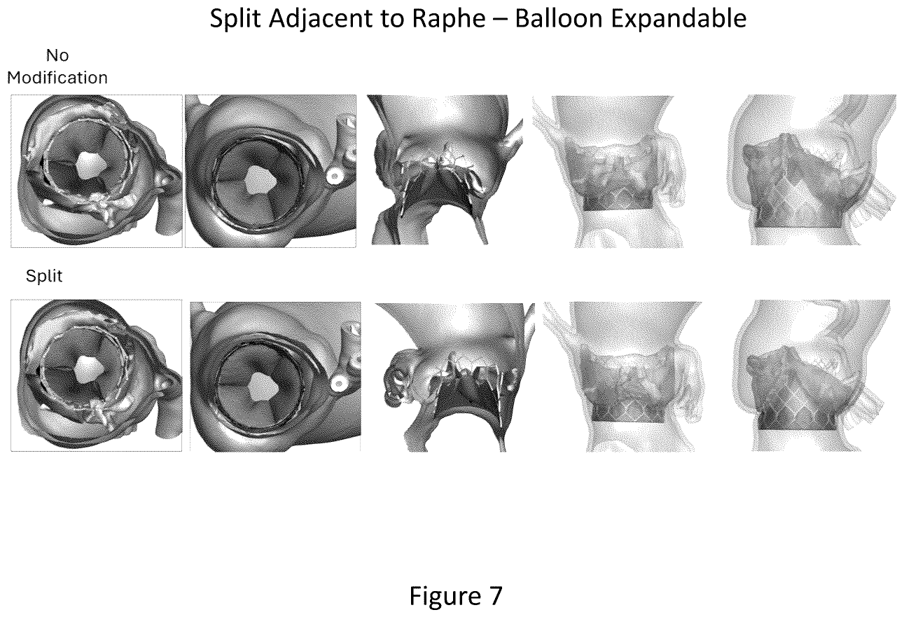

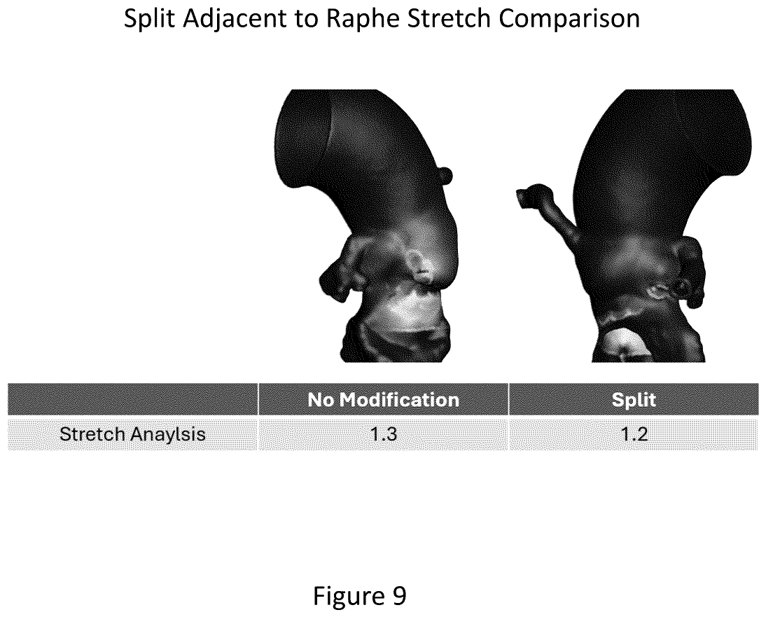

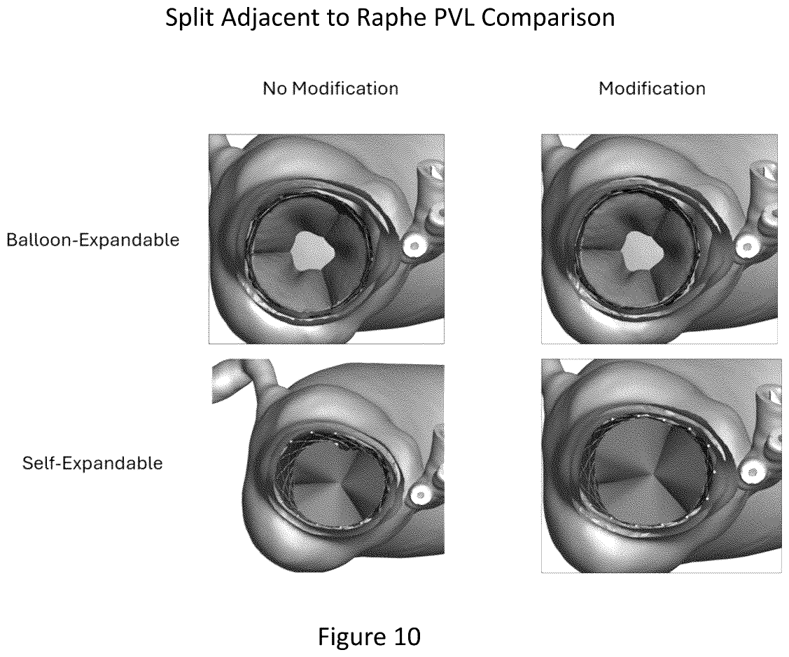

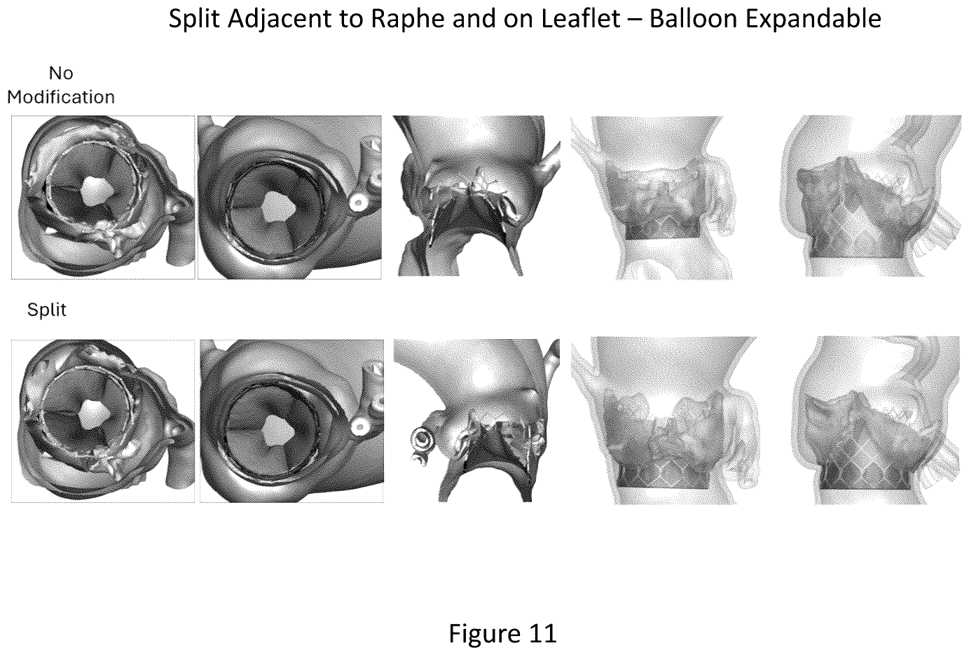

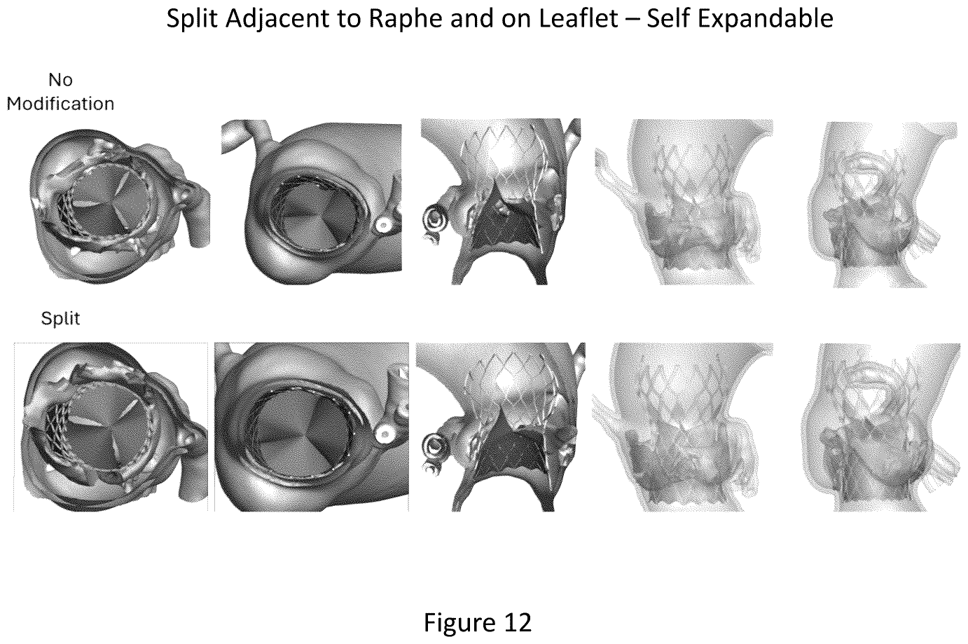

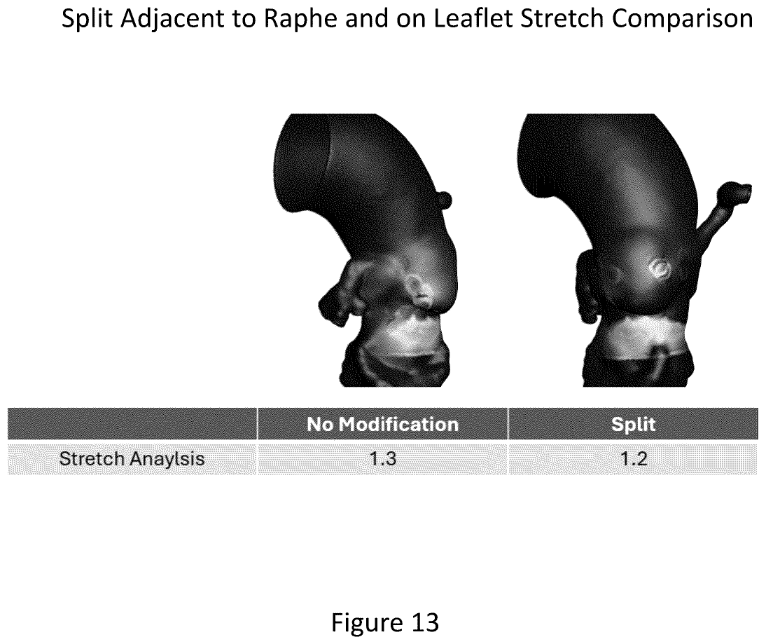

After the anatomy modifications are selected, the computer system 10 then generates the predicted valve deployments. This can be done with software available such as PrecisionTAVI™ from DASI Simulations or with finite element-based simulations. FIGS. 3-6 illustrate results of these simulations where the effect of the anatomy modification can be appreciated for the raphe splitting illustrated in FIG. 2A. It is noteworthy to see that the maximum stretch seen in FIG. 5 reduces with the split when compared to no split which is advantageous for the patient because it reduces the risk of rupture or injury to the patient. Stretch is an established measure defined as the areal stretch of the aortic tissue, as described in literature where a value of 1.6 or higher is indicative of higher risk for root or annular injury. Furthermore, FIG. 6 also demonstrates that the sealing for minimizing paravalvular leak (PVL) risk is superior with split compared to no anatomy modification. PVL risk is depicted by visually showing the gaps or frame apposition with the tissue and is measured either as size of gaps or with flow information using CFD analysis. Ideally the gaps should be zero mm for complete sealing with smaller the gaps the better. Similarly, FIG. 7-10 shows simulated results for the split adjacent to the raphe (shown in FIG. 2B). Also FIGS. 11-14 shows simulated results for the two splits shown in FIG. 2C. It is clear that comparing the stretch between the three anatomy modifications, the first raphe split yields the least stretch as well as lowest risk for PVL. The physician can use either their clinical judgement or request for the optimal modification configuration that jointly optimizes (by minimizing) stretch, paravalvular leak (PVL) gaps, and other risks. The optimization can be achieved using either a local gradient decent method or a global minimization of the total risk defined as a linear or non-linear combination of the individual risk parameters. Normalization of each risk parameter may also be utilized to condition the optimization function to reduce uneven weighting of one risk parameter over the others.

In one embodiment, the computer system 10 explicitly performs a sequential optimization pipeline comprising: (1) receiving anatomical and implantable device input datasets, (2) executing a modification-strategy generator to construct parameterized anatomy and device-modification decision options, (3) applying predictive simulations to each candidate strategy using finite-deformation models, CFD, FSI, and path-induced mechanical loading predictors as determinants of final device landmark orientation, (4) conditioning all simulation outputs using a biomarker-normalization layer that promotes equalized feature weighting, (5) quantitatively ranking all normalized options using a composite objective function that seeks coupled risk minimization, (6) selecting a ranked-optimal modification protocol, and (7) generating physician-interpretable procedural guidance outputs. This pipeline demonstrates system possession of algorithmic strategy-ranking and executable parameter selection for guiding a clinical modification workflow.

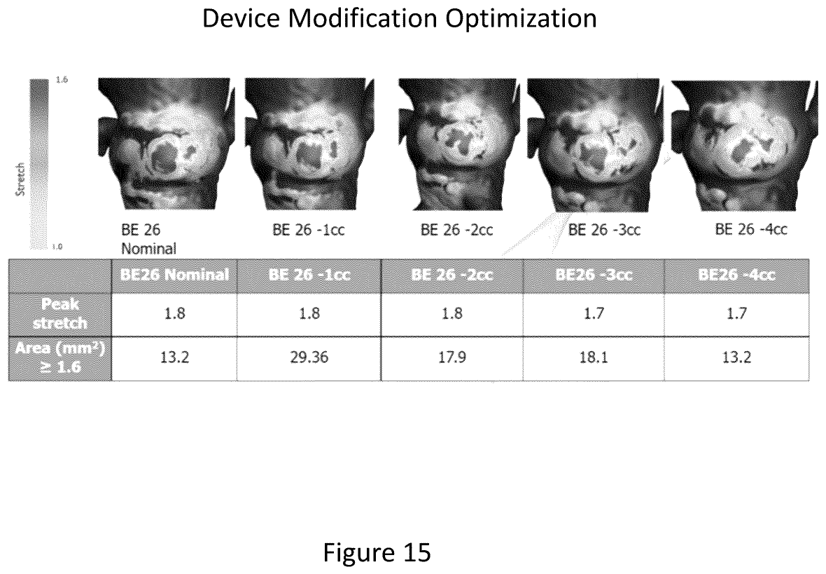

If the stretch was still high, it is further possible to optimize the device by altering the balloon inflation volume (device modification). FIG. 15 illustrates simulation results on a different bicuspid patient case where the physician is exploring the reduction of balloon inflation volume from no reduction all the way to 4 cubic centimeter (cc) reduction in volume at increments of 1 cc. Here it can be seen that the peak stretch is reduced with more reduction in balloon inflation volume. This is an example of device modification. It is further possible for device modification to also have a delivery system 30 with a sensor system 40, e.g., a pressure sensor or other sensors incorporated in the balloon or the mechanical or bioprosthetic heart valve itself. In certain embodiments, the system incorporates one or more miniature position or expansion sensors configured to monitor the radial enlargement of a transcatheter heart valve stent during balloon-mediated deployment. The sensors may be integrated directly onto the stent frame, embedded within a balloon or catheter structure, or positioned adjacent to the stent to measure local or global deformation. In one embodiment, thin-film resistive strain sensors are deposited onto selected stent struts. A thin insulating layer, such as parylene or polyimide, is applied to the metallic stent surface, followed by deposition and patterning of metallic serpentine elements whose electrical resistance varies proportionally with mechanical strain. Encapsulation layers protect the sensor while micro-scale conductive traces route signals to a proximal interface. As the balloon expands the stent, the strain induced in the underlying strut produces a measurable resistance change that corresponds to local or overall stent diameter.

In another embodiment, capacitive sensors are used to detect relative displacement between surfaces. Interdigitated electrodes may be printed or sputtered onto the inner surface of the balloon, the outer surface of the stent, or onto adjacent stent struts. Variations in separation during expansion produce quantifiable changes in capacitance, which may be monitored by a capacitance-to-digital converter in the delivery system. Similar embodiments include expandable sleeves incorporating printed piezoresistive or piezo-capacitive traces that stretch with the stent, thereby providing a continuous measurement of circumferential strain without altering permanent stent structure.

In yet another embodiment, one or more optical fiber sensors, such as Fiber Bragg Gratings (FBGs), are positioned along the stent or catheter. Strain induced during radial expansion produces a wavelength shift in the reflected optical signal, enabling highly sensitive real-time measurement of stent deformation. A single fiber may host multiple gratings, allowing distributed sensing along the stent length. Alternative embodiments utilize miniature magnetic or Hall-effect sensors, wherein micro-magnets affixed to stent nodes interact with Hall-effect devices mounted on the balloon or catheter. Changes in radial separation during expansion alter the detected magnetic field, providing a direct measure of stent diameter. In other embodiments, ultrasonic transducers—such as piezoelectric or CMUT devices—emit radial pulses and measure the time-of-flight of echoes returning from the metallic stent frame. This enables reconstruction of the inner lumen profile as the stent expands, without requiring any modification of the stent itself.

Further embodiments employ inductive or eddy-current sensing, where micro-coils in the catheter detect impedance variations caused by the metallic stent as its geometry changes. Additional embodiments may use radiopaque, optical, or reflective markers placed on the stent or balloon, imaged by a miniature fiber-optic or chip-based camera to track marker separation using image-processing algorithms. Each of these sensing modalities may be used independently or in combination to provide continuous, real-time, multi-point assessment of stent expansion, eccentricity, and deployment uniformity. The sensors may be single-use and removed with the delivery system after the procedure, or certain embodiments may incorporate permanent sensors for post-deployment monitoring.

The computer system 10 could also guide the balloon expansion of the device through a robotic platform 42 and adjust the volume in real-time in response to the sensor readings such as the pressure in the balloon. In certain embodiments, the robotic platform utilizes the above-described sensor system 40 to execute a closed-loop, sensor-guided deployment of the transcatheter heart valve prosthesis. As the stent is expanded—either through automated balloon inflation or controlled sheath retraction—the miniature sensors positioned on or adjacent to the stent provide continuous real-time measurements of local strut strain, radial displacement, stent-balloon interaction, lumen diameter, or magnetic, optical, or ultrasonic signatures indicative of the instantaneous expansion profile. These sensor outputs are transmitted to a robotic controller 44 of the robotic platform 42, which processes the data using one or more algorithms configured to estimate prosthesis eccentricity, degree of expansion, annular conformity, axial depth, rotational alignment, and proximity to critical anatomical landmarks such as the coronary ostia. Based on these measurements, the robotic module dynamically adjusts actuator commands for catheter translation, rotational orientation, balloon pressurization, or sheath retraction rate to correct deviations from the optimal deployment trajectory. For example, if the sensors detect asymmetric expansion, under-expansion at a particular segment, excessive radial force, or drift relative to the predetermined annular position, the robotic system automatically modifies its actuation profile by slowing or pausing expansion, performing micro-adjustments in axial or rotational positioning, or altering inflation pressure until anatomical and expansion metrics fall within acceptable thresholds. In this manner, the robotic platform uses direct stent-level feedback to iteratively guide deployment toward an optimal, symmetric, and anatomically aligned final position, thereby reducing operator variability and improving procedural safety and accuracy. The computer system 10 could be trained to detect and adjust the balloon inflation rate and target volume based on the training data as well as real time sensor measurement during the actual procedure.

In some aspects, the present disclosure collectively combine the incorporative robotic actuation to assist deployment, the predictive-optimization control loop, strategy-ranking engine, and anatomically anchored procedural guidance, and closed-loop actuation logic configured to transform continuous sensor or predicted deformation outputs into ranked procedural correction pathways.

It is possible that the final strategy for this case is both device and anatomy modification. Based on the outputs of computer system 10 proposed in the present disclosure, the physician would actually perform a procedure where they split the raphe and reduce the balloon volume to achieve the best possible outcome for this bicuspid patient. In some embodiments, the computer system 10 can generate step by step process for the physician or be coupled with intraprocedural imaging to help with on-screen guidance to perform these anatomy and device modification steps. The physician could utilize a cutting device like Pi-Cardia's ShortCut™ or electrosurgical techniques to achieve the optimal splits with onscreen guidance. These could be done in conjunction with device modification simultaneously.

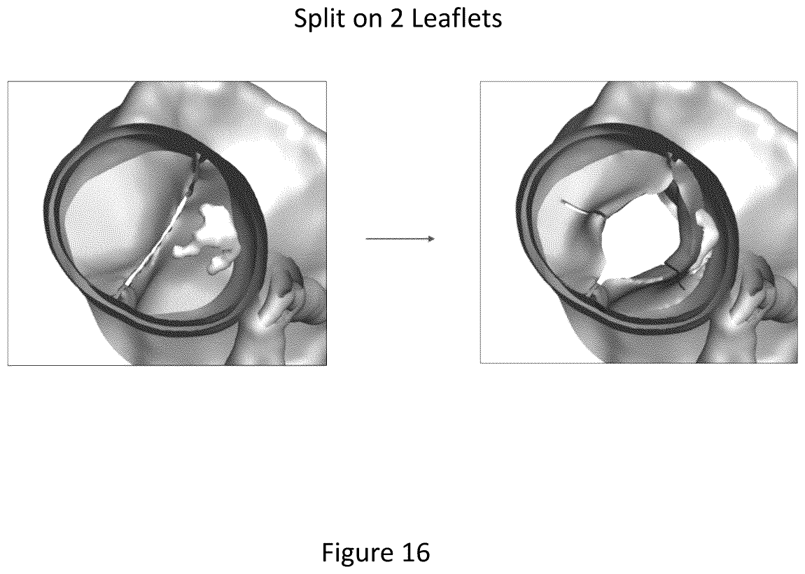

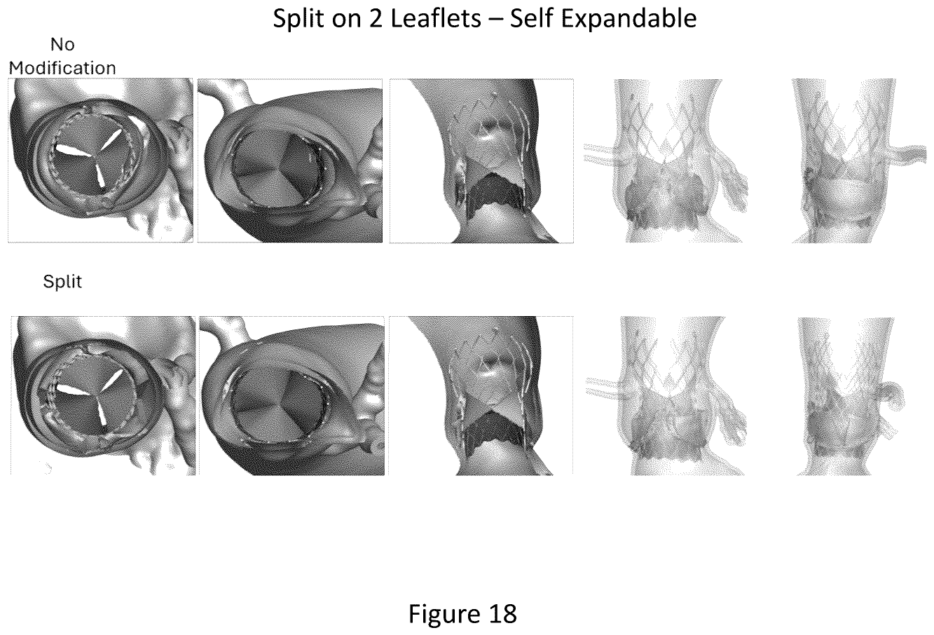

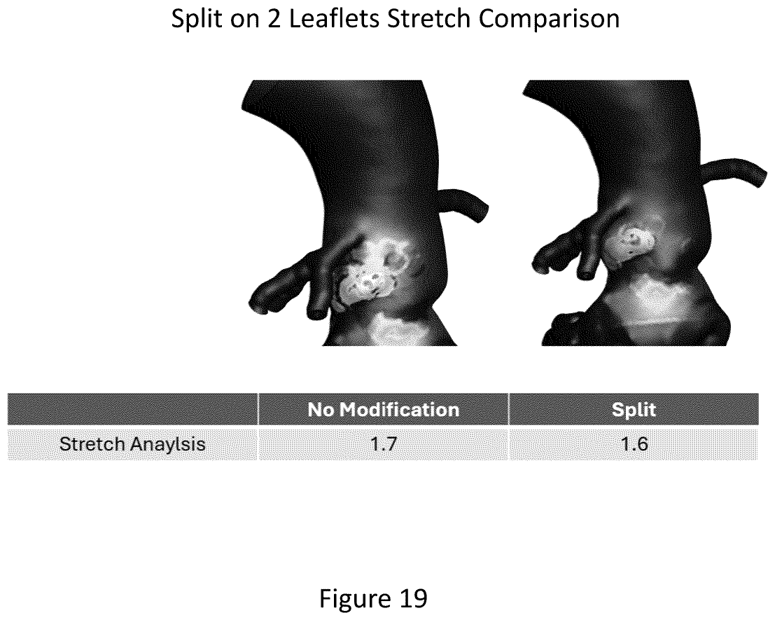

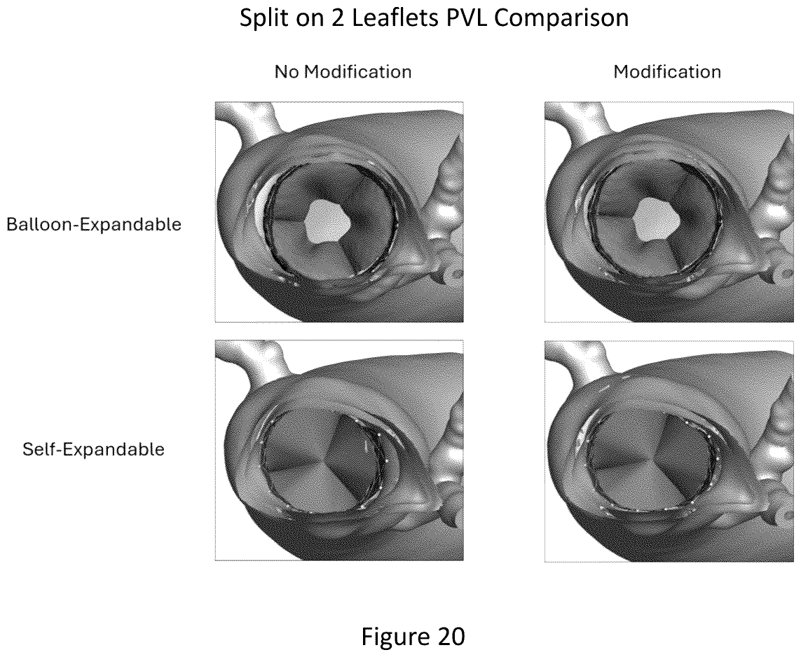

FIGS. 16-20 show simulated results of another bicuspid patient case where this case is a type 0 bicuspid and the physician is exploring cutting both the leaflets. In this scenario not only did the valve stent become more circularized with the introduction of the leaflet cuts, it also reduces the stretch significantly as seen in FIG. 19.

The computer system 10 can be further used to predict improvements in valve gradient, reduction in the potential for valve thrombosis, and longer durability of the valve replacement compared to no anatomy or device modifications. In this example, the computer system 10 is demonstrated that can be used interactively to explore anatomy and/or device modification as part of the interventional procedure itself. The method of treating the disease by the physician itself may be modified with the introduction of the computer system 10 (interactive anatomy and device modification predictive modeling tool) to be able to deliver more optimized surgical procedures.

### Example #2: Optimization for Coronary Alignment of Transcatheter Heart Valve

In some anatomies there is a need to modify the mechanical or bioprosthetic heart valve by loading the device in the delivery catheter such that when the device is deployed, certain cells frame (e.g., the geometric opening or the mesh-like pattern formed by the interconnected struts that make up the stent frame structure) on the stent frame align precisely with the coronary ostium. In certain patient-specific anatomies, there is a clinical need to modify the deployment orientation or local geometry of a mechanical or bioprosthetic transcatheter heart valve (THV) so that, upon implantation, selected cells of the stent frame (i.e., the geometric openings or mesh-like patterns defined by interconnected struts) are positioned in a predetermined alignment relative to the coronary ostium. Achieving this alignment can improve coronary access post-implant, reduce the likelihood of coronary obstruction, and facilitate subsequent interventions. To accomplish this, the THV can be custom-loaded into the delivery catheter in a rotational or axial orientation that differs from the manufacturer's default loading configuration. Such custom loading may involve rotating the prosthesis about its central axis prior to crimping, applying asymmetric crimping profiles, or selectively modifying the stent frame deformation pattern so that, after expansion, one or more target cells are predicted to lie within a specified angular and vertical tolerance window relative to the coronary takeoff.

In some embodiments, the computer system 10 computes these customized loading parameters based on a combination of pre-procedural imaging (e.g., CT-derived annular geometry, coronary height, sinus dimensions, and leaflet calcification distribution) and a forward model of valve expansion dynamics. The system may generate a patient-specific deployment model that simulates how variations in crimp orientation, crimp depth, stent-frame torsion, or delivery-catheter rotation influence the final orientation of individual stent cells. This simulation may incorporate device-specific mechanical behavior-including strut thickness, hinge regions, and asymmetric frame features-so that the resulting predictions reflect the true expansion characteristics of the selected THV platform.

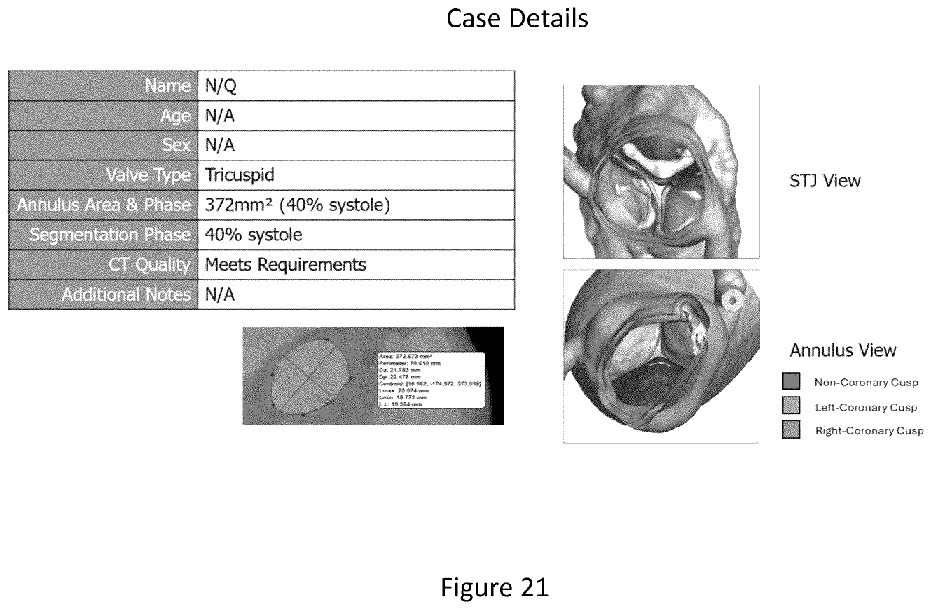

FIG. 21 illustrates one such example in which the computer system 10 is used to evaluate the Medtronic Evolut™ FX+valve. The system identifies a target aperture on the stent frame that is preferred for maintaining coronary access and then determines the optimal pre-deployment rotational offset and crimping configuration needed to position this aperture adjacent to the coronary ostium following deployment. The output may include recommended rotational markers for catheter loading, predicted angular deviation at final expansion, and quantitative metrics indicating the degree of alignment accuracy. In this way, the system enables a more customized stent-frame orientation strategy tailored to individual patient anatomy, thereby improving procedural outcomes and enabling more predictable coronary access after THV implantation.

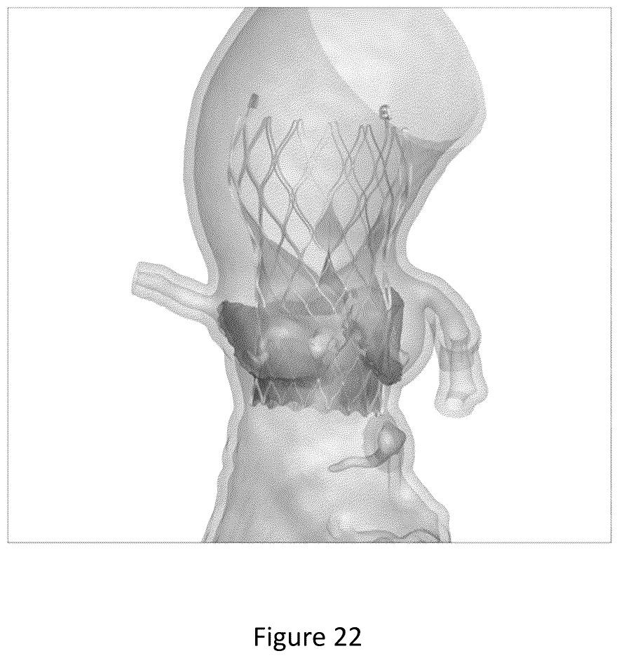

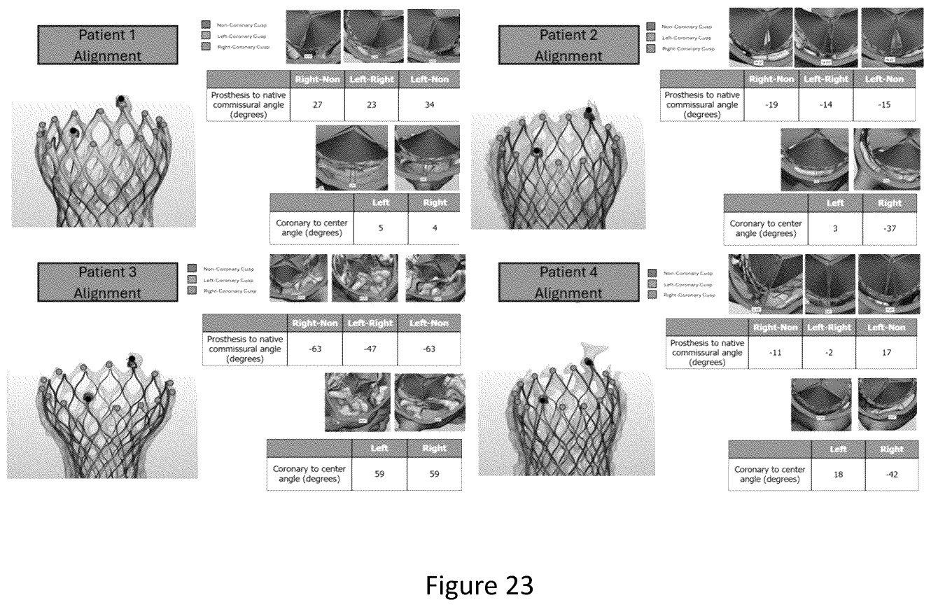

As in the previous example, the computer system 10 can predict the final deployment configuration of the transcatheter device in the anatomy. In FIG. 22, the output of the computer system 10 is interactive and can be rotated to appreciate the device/anatomy relationship by the physician. A visual display can be used to demonstrate the interaction of the device and the tissue as the device deploys. The computer system 10 generates the appropriate views of the cells in the valve and the orientation of these cells with respect to the coronary ostium as a function of how the valve is being loaded into the delivery system. Typically the loading of the valve is dependent on how the crimped valve features (such as tabs to connect to catheter) are oriented with respect to the patient's operating room table coordinate system (e.g. you can rotate the catheter or the device prior to attaching with the remaining delivery system). For balloon expandable valves the angle at which the valve is oriented prior to crimping on a balloon or the delivery catheter is another parameter. The computer system 10 utilizes computed tomography (CT) imaging data that includes the blood vessel path used by the catheter. The blood vessel path includes the ascending aorta, descending aorta, subclavian and carotid arteries, femoral arteries and the iliac arteries. The computer system 10 has been trained on previous datasets where pre and post CT scans are available where it is possible to model the loading configuration of the crimped valve on the catheter and output the final configuration of the valve. The training and loading and outputting of the heart valve configurations can be done in an interactive format). FIG. 23 shows the model training process where the post-CT reconstruction of the valve is aligned with the simulation while optimizing the predictive model that is based on the catheter path through the patient's blood vessels (femoral, iliac, descending aorta, aortic arch, and ascending aorta).

In one embodiment, a computer-implemented system 10 is provided for predicting the post-deployment configuration of an implantable heart valve based on patient-specific anatomical imaging and catheter-path-dependent mechanical loading. The system utilizes computed tomography (CT) imaging datasets that capture the three-dimensional geometry of the patient's vasculature, including, but not limited to, the ascending aorta, aortic arch, descending thoracic aorta, iliac arteries, femoral arteries, subclavian arteries, and carotid arteries. From these image datasets, the system automatically derives a centerline-based blood-vessel path corresponding to the advancement route of the delivery catheter.

The computer system 10 further comprises a predictive deformation engine trained on prior cases in which paired pre-procedural and post-procedural CT scans are available. For each of these training cases, the pre-procedural CT is used to reconstruct the vascular pathway and to model the catheter navigation path, while the post-procedural CT is used to reconstruct the final deployed configuration of the prosthetic valve. A ground-truth deformation dataset is produced by correlating the known loading configuration of the crimped heart valve mounted on the catheter with the ultimately observed expanded morphology extracted from the post-procedural CT scan.

Using these paired datasets, the system 10 trains one or more machine-learning models-such as a physics-informed neural network, finite-element-augmented neural model, graph-neural operator, or hybrid data-driven/mechanics model—to learn a mapping between (i) patient-specific catheter path-induced loading conditions applied to the crimped valve during navigation and (ii) the resulting deployed valve shape and orientation. The training framework may incorporate biomechanical simulation modules configured to apply vessel-wall contact forces, frictional effects, torsional loads, bending loads, and axial compression loads corresponding to the catheter's passage through each vascular segment.

Referring to FIG. 23, the training process involves a registration and alignment module that spatially aligns the post-procedural CT-derived valve reconstruction with the predicted valve configuration generated by the simulation engine. An optimization routine iteratively adjusts the model parameters—such as stiffness coefficients, vessel-interaction terms, hinge tolerances, and neural-network weights-to minimize the discrepancy between simulated and actual post-deployment geometries. The computer system 10 performs: [1] Extraction of vascular path geometry from the pre-procedural CT scan, [2] Reconstruction of crimped valve geometry and its mounting configuration on the catheter, [3] Simulation of catheter advancement along the segmented vessel pathway, including bending, torsion, and contact pressures imparted to the valve, [4] Generation of a predicted final valve configuration at the intended deployment site, [5] Registration of the predicted valve configuration to the post-procedural CT reconstruction, and [6] Optimization of the predictive model using error back-propagation, physics-based constraint enforcement, or hybrid optimization algorithms.

The computer system 10 thus produces a continually improving predictive model capable of accepting pre-deployment CT data only and outputting a patient-specific valve expansion prediction without requiring a post-procedural scan.

In some embodiments, the system provides an interactive visualization interface allowing clinicians to inspect the predicted deployed configuration of the heart valve in three dimensions. The interface may present: [1] Predicted stent-frame deformation patterns; [2] Rotational orientation of the valve relative to anatomical landmarks; [3] Predicted leaflet coaptation geometry; [4] Stress and strain heat maps, and [5] Potential mal-apposition or paravalvular leak zones.

The computer system 10 enables pre-procedural planning by predicting patient-specific device deformation before the device is deployed, thereby improving planning, reducing complications, and allowing selection of optimal catheter paths and valve sizes. Unlike traditional finite-element planning tools, the computer system 10 uniquely incorporates the catheter pathway through the vasculature as a determinant of final valve geometry, which has not been accounted for in conventional heart-valve deployment prediction models.

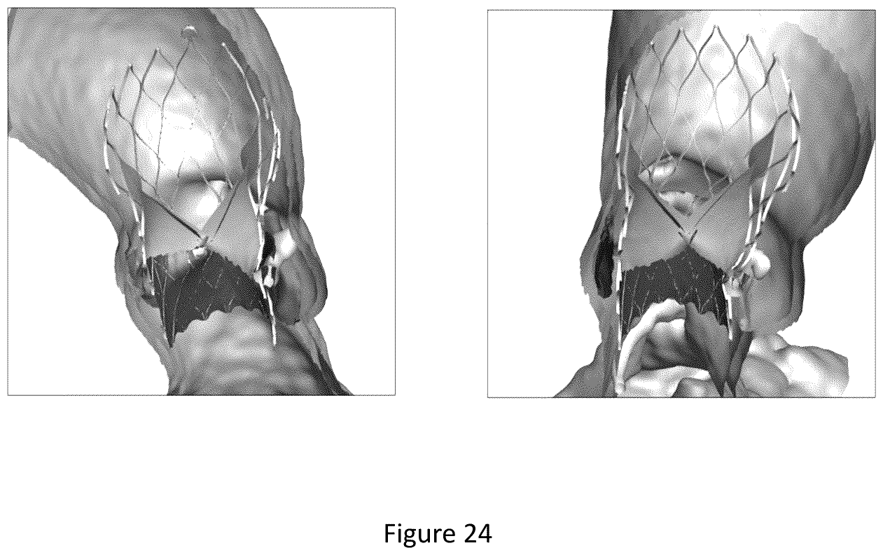

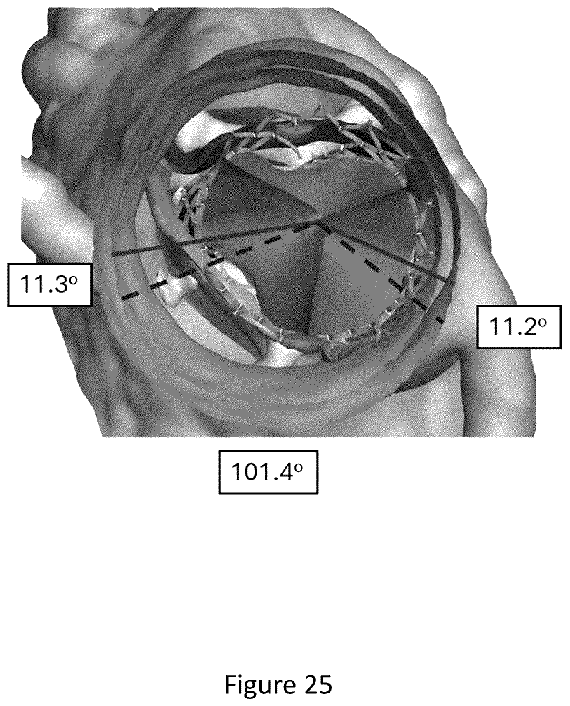

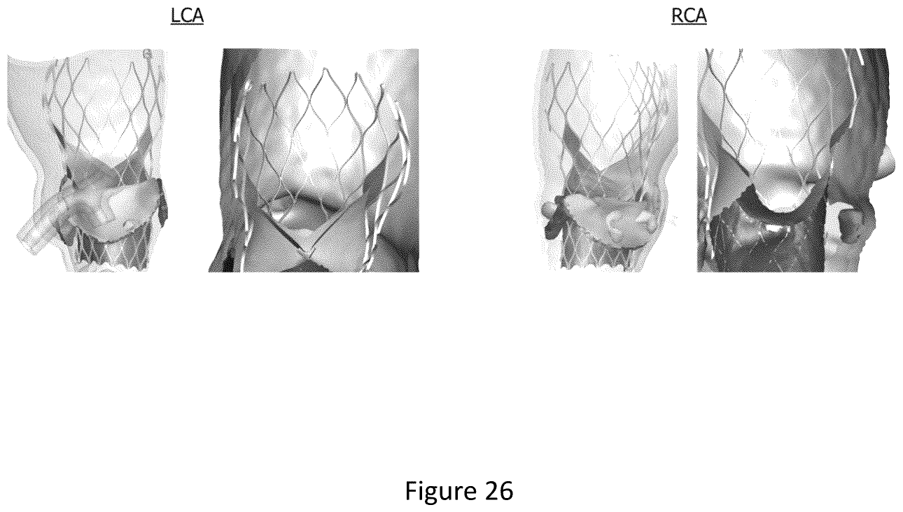

The device modification in this example is shown in FIGS. 24-26 where the interactive display of the interactive graphical user interface 20 shows the orientation of the valve and the anatomy with the display of the aperture such that such that the physician can adjust the loading angle of the valve in the catheter and receiving an in-situ (in real-time) corresponding final deployment alignment simulated by the computer system 10. The simulated final deployment alignment is communicated to the physician via the interactive graphical user interface 20. The computer system 10 is also capable of simulating device modifications including the depth optimization or any post deployment device modification such as the use of a post dilatation balloon. FIG. 25 also gives simulated optimal angles with respect to the coronaries for the patient case where the coronary angulation is displayed (e.g. 101.4°).

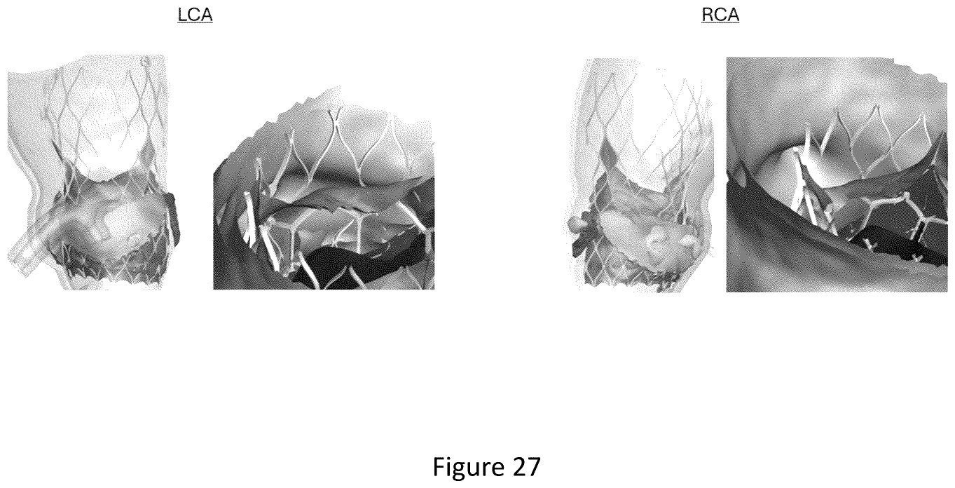

In FIG. 27, the computer system 10 is used to help with device optimization with respect to future intervention as it relates to coronary access and alignment of the heart valve cells optimized for the specific patient. Further, the computer system 10 is interactive to allow the physician to load a simulated future balloon-expandable valve to view how that future procedure may or may not interfere with coronary access for the given alignment of the first valve.

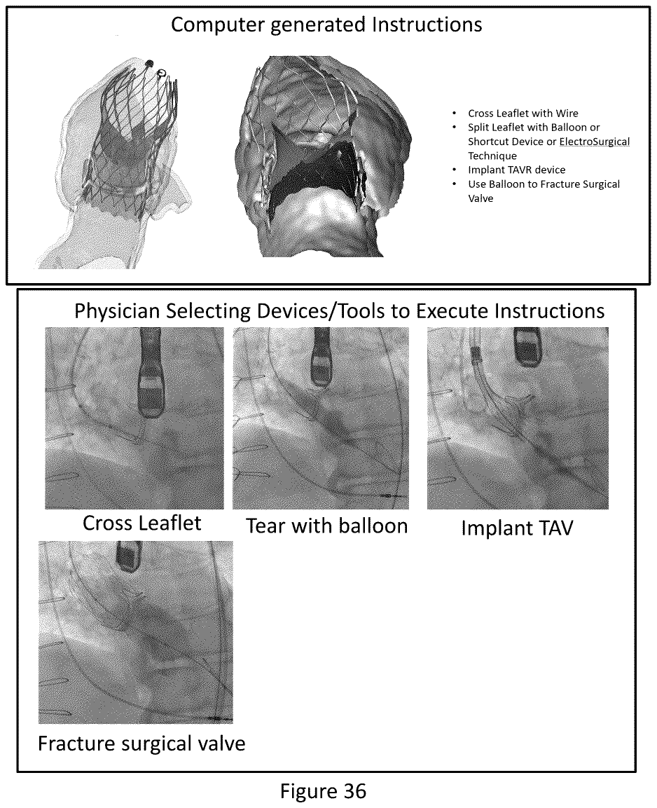

In FIG. 36, The figure illustrates a system and physician-implemented method for computer-assisted modification of a previously implanted, degenerated or structurally failed surgical heart valve to improve transcatheter aortic valve replacement (TAVR) deployment and post-procedure hemodynamic performance. The upper portion of the figure depicts computer-generated modeling output including a three-dimensional rendering of the failed surgical valve, overlaid structural elements corresponding to one or more device-optimization instructions, and a text-based workflow of sequential modification steps determined by a computational planning system. The planning system analyzes the geometry and mechanics of the failed surgical valve and algorithmically proposes steps for enlarging the effective orifice area, reducing transvalvular gradient, and optimizing anchoring and expansion of a TAVR prosthesis whole reducing the risk for coronary occlusion by prescribing methods to modify via splitting of the leaflet.

The sequence of modeling-derived steps shown in the figure includes: (1) traversal of the compromised valve leaflet using a guide element such as a wire passed across and through a target leaflet; (2) division or splitting of the leaflet using at least one of balloon expansion, an electrosurgical energy delivery technique, or a dedicated leaflet-splitting shortcut device; (3) implantation of a TAVR device within the modified surgical valve frame, exemplified in the figure by an implanted transcatheter valve prosthesis corresponding to an Evolut FX+deployment; and (4) expansion of a high-pressure balloon within the previously implanted surgical valve structure to induce controlled fracture of the surgical valve frame or ring to increase valve opening.

The lower portion of the figure depicts real-time execution of the planned method by a physician in a catheterization laboratory or hybrid operating room. This section schematically demonstrates that the physician interprets the modeling instructions and performs the corresponding valve-modification workflow in vivo, including selection of specific endovascular tools to achieve the planned optimization steps. The illustrated physician-selected tool sequence includes: a 10 mm dilation balloon for mechanically splitting the targeted failed leaflet, followed by implantation of the self-expanding Evolut FX+transcatheter valve prosthesis within the modified surgical valve environment, and subsequent delivery of a larger, high-pressure balloon having a 22 mm nominal diameter to apply radial force sufficient to fracture the pre-existing surgical valve frame, thereby expanding the flow aperture, lowering pressure gradient, and improving overall TAVR hemodynamic outcome.

The figure thus conveys an integrated system in which computational modeling produces patient-specific or valve-specific optimization instructions for surgical valve modification, and further shows a method of physician-directed procedural execution incorporating intra-procedure device and technique choices aligned with the computer-generated plan. The illustration supports the full claimed method including: digital generation of a structural optimization sequence, physician review and interpretation of the generated plan, trans-leaflet crossing using a guide member, leaflet division using a selected balloon or energy-based or shortcut splitting modality, TAVR prosthesis implantation such as with the Evolut FX+device, and controlled fracture of a failed surgical valve using a physician-selected high-pressure balloon to achieve improved TAVR performance.

The above example while depicted for device optimization can also be illustrative of a physician executed method for both anatomy and device optimization execution intra operatively, either with real time guidance or without real time guidance from sensor feedback (if the devices or delivery mechanisms have sensors described previously). Any combination of a computer implemented method combined with a physician tool to help achieve the modeled method to optimize will be the subject of the method disclosed in these examples.

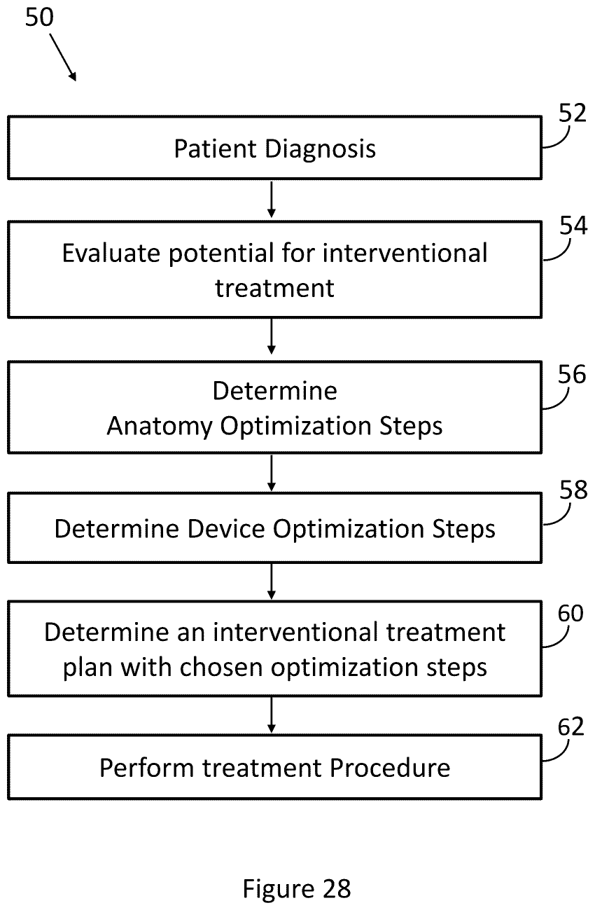

FIG. 28 shows an example computer-implemented method 50 using the computer system 10 shown in FIGS. 30A and 30B for a heart procedure. The method 50 includes providing a patient diagnosis (step 52), evaluating potential for interventional treatment (step 54), determining anatomy optimization steps (step 56), determining device optimization steps (step 58), determining an interventional treatment plan with chosen optimization steps (step 60), and performing treatment procedure (step 62).

With reference to step 52, the patient diagnosis may be obtained from CT and echocardiography imaging. Step 52 may include obtaining data related to a heart medical procedure for a patient (e.g., a transcatheter aortic valve replacement (TAVR), a valve-in-valve (ViV) replacement, a surgical valve replacement, etc.). The data may include patient-specific data including 3D computational analysis generated from pre-procedural medical imaging. The data may include device data related to at least one device to be deployed in the heart medical procedure (e.g., the device referenced herein may be a heart valve, a guidewire, a cutting device, an electrosurgical splitter, a catheter, a deployment system of the at least one device, and/or a left atrial appendage occlusion device). The data may include interactive input from medical personnel (e.g., a physician).

The data may include data communicated in real-time during the heart medical procedure from a sensor system comprising one or more sensors incorporated in the at least one device or a deployment system of the at least one device. The patient-specific data comprises at least one of: native valve morphology, physicologic conditions, patient demographics, structural measurements of aortic root, annulus, sinotubular junction, coronary heights, leaflet calcification volume and distribution dynamic physiologic parameters, and the pre-procedural medical imaging data from CT, MRI, echocardiography (TTE or TEE), fluoroscopy, or catheter-derived hemodynamic recordings.

Step 54 includes generating, based on the data, options of modification strategies including device modifications to the at least one device and anatomical modifications to the patient's anatomy. It also includes simulating the heart medical procedure and generating a simulation outcome for each option of the modification strategies. The simulation may be done through a predictive simulation using one or more computational models selected from: nonlinear finite element analysis (FEA), fluid-structure interaction (FSI) modeling, computational fluid dynamics (CFD), reduced-order surrogate models, simplified force-driven particle models, and machine-learning-based risk estimation models. The simulation outcome includes ranking the modification strategies with quantitative justification and valve performance for each modification strategy. Quantitative risk metrics may be used to evaluate outcomes of the modification strategies. The medical personnel or physician inputs may be taken into account at this step. Step 54 may include displaying the procedural recommendations via a visual display capable of allowing the medical personnel to provide interactive input. Such interactive input may be communicated to the computer system 10 any time (not limited to step 54).

Based on evaluations in step 54, in steps 56 and 58, the computer system 10 can select a modification strategy that corresponds to the simulation outcome having a desired heart medical procedure result and determine an interventional treatment plan (step 60). The computer system 10 can generate procedural recommendations including device deployment instructions and modification parameters to guide the heart medical procedure in real life. A physician can perform the treatment procedure based on the detailed plan recommended by the computer system 10. In some embodiments, the computer system 10 may perform treatment procedure at least by generating a selection of at least one modification device (e.g., a cutting device, a splitter, etc.) for the selected modification strategy. In some embodiments, the computer system 10 may even be operatively coupled to the robotic platform 42 to facilitate operation of the selected modification strategy. For example, the computer system 10 could guide the balloon expansion of the device through the robotic platform 42 and adjust the volume in real-time in response to determination of the anatomy and device optimization strategies. Herein examples of anatomical modifications may include but not limited to splitting, releasing, incising, thinning, scoring, and/or debulking to alternate native leaflets, commissures, raphe tissue, and calcified regions. Examples of device modifications may include but not limited to structural alterations to the at least one device, size or dimensional adjustments of the at least one device, orientational adjustments of the at least one device, alignment adjustments of the at least device, balloon-related modifications for a balloon-expandable implant device, adding sealing elements to a leaflet and/or a skirt of the at least one device.

In some embodiments, catheter-path mediated implant deformation is included as a pre-deployment optimization variable within the generative modification strategy pool. The computer system 10 derives a patient-specific vascular centerline path from pre-procedural imaging and applies path-induced mechanical loading estimates to the crimped implant model to generate a predicted deformation state prior to expansion. Path-dependent loading variables, comprising torsional, bending, axial compression, frictional, or vessel-wall contact interactions, become parameterized predictors that influence candidate strategy generation. These deformation predictors are evaluated by the simulation engine as part of the optimization decision space and quantitatively ranked with other biomarkers for selecting anatomical or device-orientation modifications to reduce predicted complication risk and improve final landmark alignment.

In some embodiments, the 3D computational analysis comprises incoming CT or MRI DICOM image stacks segmented using a machine-implemented tissue-segmentation model to produce a patient-specific digital twin including the valve annulus, native leaflets, sinuses, coronary ostia centerlines, and calcific nodules. The segmentation engine generates a finite-element mesh defined by vertex positions, element connectivity, and material tags, with spatial fidelity sufficient to resolve anatomical features at ≤0.5 mm and leaflet tip thickness at ≤0.25 mm. Candidate anatomy-modification strategies are parameterized as node-editing operations over the mesh, including laceration paths modeled by sequential removal of mesh elements across a spline-defined trajectory, or tissue-thinning regions modeled by localized thickness field reduction, each stored as parametric action vectors. Candidate device-modification strategies are represented as pre-crimping configuration parameters (rotational crimp-axis offset, asymmetric crimp depth profiles, or stent-torsion state), catheter rotation parameters, or balloon-expansion parameters (target inflation volume, inflation rate curve, or expansion depth). Biomarkers computed by the numerical analysis engine comprise: (i) coronary obstruction gap distance defined as the minimum Euclidean distance between any deployed stent-frame cell edge and the coronary ostium centerline, (ii) paravalvular leak (PVL) gap size defined as the maximum 3D radial distance between the expanded stent frame and the annulus sealing interface, and (iii) tissue injury stretch surrogate defined as peak areal or radial stretch fields over annular or sinus tissue under simulated deployment. Each biomarker is normalized prior to ranking using feature-wise min-max or z-score scaling implemented in a biomarker-conditioning layer that bounds uneven metric influence. Normalized candidate strategies are ranked using a composite objective function that performs coupled risk minimization assigning equalized or configurable weighting per biomarker. Real-time execution is enabled by streaming intraoperative sensor inputs sampled at ≥10 Hz, including radial expansion strain, pressure feedback, or anatomical progress indicators, which feed a closed-loop inference engine that recomputes and re-ranks the candidate decision space in ≤500 ms per update cycle to maintain intraoperative responsiveness. Procedural recommendations are output as clinician-interpretable guidance packets comprising deployment orientation markers, leaflet-modification paths, and inflation or crimping parameters, transmitted to a display interface or proximate control module for visual or encoded instruction to assist physician-performed intervention.

The steps 52-62 discussed above may be implemented in-situ, in real-time during a heart medical procedure.

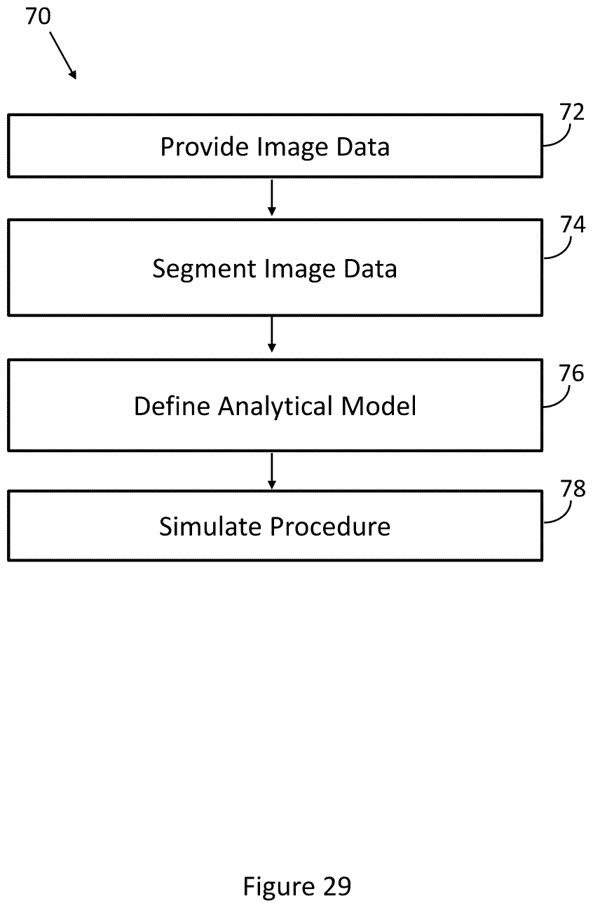

FIG. 29 shows an example computer-implemented method 70 using the computer system 10 show in FIGS. 30A and 30B for patient diagnosis (step 52 in FIG. 28) and evaluating potential for interventional treatment (step 54 in FIG. 28). The method 70 may include providing image data (step 72), segmenting image data (step 74), defining analytical model (step 76), and simulating procedure (step 78).

With reference to steps 56 and 58 in FIG. 28, once the modeling of the THV deployment in a patient's pre-existing anatomical condition is completed, various computationally derived biomarkers can be generated and analyzed to determine the risk of various complications during the TAVR procedure. These include but are not limited to coronary obstruction, paravalvular leak, aortic root rupture, pacemaker implantation, etc). Once the baseline risk thresholds for these complications are determined, additional simulations, representative of device or anatomical modifications, can be performed in order to demonstrate demonstrated to the clinician how certain modification techniques can be used to mitigate or reduce the severity of these procedural modifications.

FIGS. 30A and 30B illustrate exemplary computer system configurations, and the computer system 10 can be utilized to execute any aspects of the components and/or modules presented herein described as executing on the analysis system or any components in communication therewith. The computer system 10 can be configured to collect image data characterizing a heart of a patient 80. The computer system 10 can include an imaging device 100. The imaging device 100 can be configured to college image data 82 in two or three dimensions of the patient. The image data 82 can include, but not limited to, X-ray image data (e.g., X-ray computed tomography (CT) images), magnetic resonance imaging (MRI) image data, or ultrasound image data. An imaging device 100, as described herein, can correspond to any modality that can be configured to collect image data 82 of the patient 80, such as the patient's heart.

The imaging device 100 can further include one or more processors 102 for executing machine readable instructions and memory 104 for storing the machine readable instructions. The one or more processors 102 can be coupled to the memory 104, and configured to retrieve the stored machine readable instructions at the memory 104. The one or more processors 102 can include an integrated circuit, a microchip, a computer, or any other computing device capable of executing machine readable instructions. The memory 104 can include RAM, ROM, a flash memory, a hard drive, or any device capable of storing machine readable instructions.

The imaging device 100 can further include a sensor 106 and the sensor system 40. The sensor 106 can be configured to collect measurements of the heart of the patient 80. The sensor system 40 can be configured to collect data or measurements from the balloon or the heart valve itself, including but not limited to pressure measurements, radial force, flow velocity, shear stress, stent-frame deformation, balloon compliance, leaflet motion, transvalvular pressure gradients, coronary flow changes, and other hemodynamic or mechanical parameters relevant to valve performance and procedural safety. The sensor 106 and the sensor system 40 can be coupled to the one or more processors 102, the memory 104, or both. It is noted that the term “sensor,” as used herein, corresponds to a device that can be configured to measure a physical quantity and convert the measured physical quantity into a representative signal, which can be correlated to a measured value of the physical quantity. In some examples, the imaging device 100 can include an X-ray CT system for collecting X-ray data. Accordingly, the sensor 106 can be an X-ray detector, and can be configured to detect photons such as, for example, a point detector, a linear detector, or a planar detector. Accordingly, the sensor system 40 can include a pressure sensor . . . .

In some examples, the imaging device 100 can include a source 108. The source 108 can be configured to generate excitation energy that can be detectable by the sensor 106. The source 108 can be coupled to the one or more processors 102, the memory 104, or both. In examples where the imaging device 100 includes an X-ray CT system, the source 108 can be an X-ray source. The X-ray can be configured to emit photons along a path. The path can begin at the source 108 and terminate at the sensor 106. The heart of the patient 80 can be located along the path, and thus between the source 108 and the sensor 106. A portion of the photons can be absorbed by the patient 80, while measurements are collected by the sensor 106. Accordingly, the photons received by the sensor 106 can be indicative of the patient 80, e.g., the intensity of the photons can be correlated to the density of patient's 80 body.

The imaging device 100 can further include an actuation assembly 110. The actuation assembly 110 can be configured to manipulate the patient 80, the sensor 106, the source 108, or a combination thereof. For example, the actuation assembly 110 can include one or more servo-mechanisms that can be configured to control an amount of force required for manipulating the patient 80, the sensor 106, the source 108, or a combination thereof. In the examples described herein, the one or more processors 102, the memory 104, or both can be integral with any or all of the sensor 106, the source 108, and the actuation assembly 110. However, it is to be understood that the one or more processors 102, the memory 104, or both, can be separate components that can be coupled with one another.

In some examples, the actuation assembly 110 can include a mechanical actuator, a hydraulic actuator, a pneumatic actuator, an electrical actuator, or a combination thereof. The actuation assembly 110 can be coupled to the one or more processors 102, the memory 104, or both. The one or more processors 102 can be configured to execute the machine readable instructions to control the operation of the sensor 106, the source 108, and the actuation assembly 110. The actuation assembly 110 can be configured to cause relative motion of the patient 80 with respect to the sensor 106 and the source 108. For example, the actuation assembly 110 can include a gantry system for moving the sensor 106 and the source 108 in a substantially circular pattern relative the patient 80.

In examples where the imaging device 100 includes an X-ray CT system, multiple measurements of the patient 80 can be collected by the sensor 106, relative motion between the patient 80 and the sensor 106, the source 108, or both. Each measurement can be constructed into an image having greater dimensional complexity than the measurement generated by the sensor 106. For example, each measurement can be indicative of absorption or density of the patient 80, and can be constructed into the image data 82 indicative of the anatomy of the patient 80. For example, measurements collected by a line detector can be used to produce a two-dimensional images showing a slice of the patient's anatomy. A plurality of slices can be combined to provide a full representation of the patient 80 in three-dimensions such as, for example, by combining slices collected along a direction orthogonal to the plane of the slices. Measurements collected by a planar detector can be combined into three-dimensional images of the patient 80.

The imaging device 100 can further include network interface hardware 112. The network interface hardware can be coupled to the one or more processors 102 such that the imaging device 100 can be coupled to another device via a network. The network can include, but not limited to, a wide area network (WAN), a local area network (LAN), a personal area network (PAN), or a combination thereof. The network interface hardware 112 can be configured to communicate (e.g., send and/or receive data signals) via any wired or wireless communication protocol. For example, the network interface hardware 112 can include an antenna, a modem, LAN port, wireless fidelity (Wi-Fi) card, WiMax card, near-field communication hardware, or the like. Accordingly, the imaging device 100 can be coupled to a network via wires, a WAN, a LAN, a PAN, or the like.

Suitable LANs can include, but not limited to, wired Ethernet and/or wireless technologies such as, for example, Wi-Fi. Suitable PANs can include, but not limited to, wireless technologies such as, for example, infrared data association (IrDA), BLUETOOTH, wireless universal serial bus (USB), Z-WAVE, ZIGBEE, or the like. Alternatively or additionally, suitable PANs can further include, but not limited to, wired computer buses such as, for example, USB and FIREWIRE. Thus, any components of the imaging device 100 can utilize one or more network components to communicate data via the network.

The computer system 10 can further include an image analysis device 200. The image analysis device can be configured to executing machine readable instructions to provide image analysis and anatomical simulation functionality based on anatomical information extracted from the image data 82. The image analysis device 200 can include one or more processors 202. The one or more processors 202 can be configured to retrieve and execute the machine readable instruction stored in memory 204. The one or more processors 202 can be coupled to network interface hardware 206. It is noted that, while the image analysis device 200 is illustrated in the example of FIG. 30A as being a single machine, each of the one or more processors 202, the memory 204, and the network interface hardware 206, including their components and functions, can be distributed amongst a plurality of machines that can be communicatively coupled to one another. Additionally, it is noted that in some examples, the image analysis device 200 and the imaging device 100 can be implemented on a single machine. The embodiments of the present disclosure can include logic, processes, or an algorithm written in any programming language of any generation (e.g., 1GL, 2GL, 3GL, 4GL, or 5GL) such as, e.g., machine language that can be directly executed by the processor, or assembly language, object-oriented programming (OOP), scripting languages, microcode, etc., that can be compiled or assembled into machine readable instructions and stored on a machine readable medium. Alternatively, the logic, processes, or algorithm can be written in a hardware description language (HDL), such as implemented via either a field-programmable gate array (FPGA) configuration or an application-specific integrated circuit (ASIC), and their equivalents.

The image analysis device 200 can further include a display 208. The display 208 can be coupled to the one or more processors 202. Alternatively or additionally, the display can be provided as a wearable device, such as, for example a smart watch or a virtual reality headset. Suitable example of virtual reality headsets can include Samsung Gear VR, Sony PlayStation VR, Oculus Rift, or the like.

FIG. 30B shows another exemplary computer architecture for the computer system 10 capable of executing the software components that can use the output of the exemplary method described herein. The computer system 10 may include a computing device 300 which can be utilized to execute any aspects of the components and/or modules presented herein described as executing on the analysis system or any components in communication therewith. The computer device 300 may be an integral part or a distinctly separate part from the systems shown in FIG. 30A.

In an aspect, the computing device 300 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an aspect, virtualization software may be employed by the computing device 300 to provide the functionality of a number of servers that is not directly bound to the number of computers in the computing device 300. For example, virtualization software may provide twenty virtual servers on four physical computers. In an aspect, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

The computing device 300 may include some or all of the engines and/or modules in the present disclosure contributing to anatomical and device optimization. For example, in one embodiment, the computer system 10 including the computing device 300 includes an executable optimization architecture (e.g., a module or engine 310) that includes a modification strategy generator module, a simulation execution engine, a biomarker normalization layer, a quantitative ranking comparator, and a strategy selector. The modification strategy generator module algorithmically constructs a candidate decision space that comprises: (i) anatomical remodeling actions selected from tissue splitting, incising, releasing, debulking, scoring, thinning, or laceration placement parameterizations, and (ii) device configuration modifications selected from crimp-rotation offsets, balloon inflation adjustments, axial-depth offsets, yaw or pitch alignment parameters, or sealing augmentation profiles. The simulation execution engine applies predictive models to each candidate option to generate structural and hemodynamic outcomes. The biomarker normalization layer conditions quantitative outputs by applying feature-wise scaling to measured biomarkers comprising coronary obstruction gap size, LVOT (left ventricular outflow tract) stretch or strain injury surrogates, and frame-tissue malapposition distances that correlate to paravalvular leak. The quantitative ranking comparator evaluates all normalized candidate outcomes using a composite objective function that assigns equalized weighting to each biomarker to prevent uneven risk influence. The strategy selector identifies an optimal modification protocol based on the ranked results and outputs procedural recommendations configured to guide clinical execution.

In its most basic configuration, computing device 300 typically includes at least one processing unit 320 and system memory 330. Depending on the exact configuration and type of computing device, system memory 330 may be volatile (such as random-access memory (RAM)), non-volatile (such as read-only memory (ROM), flash memory, etc.), or some combination of the two. The processing unit 320 may be a standard programmable processor that performs arithmetic and logic operations necessary for operation of the computing device 300. While only one processing unit 320 is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUS), and application specific circuits (ASICs). Thus, while instructions may be discussed as executed by a processor, the instructions may be executed simultaneously, serially, or otherwise executed by one or multiple processors. The computing device 300 may also include a bus or other communication mechanism for communicating information among various components of the computing device 300.

Computing device 300 may have additional features/functionality. For example, computing device 300 may include additional storage such as removable storage 340 and non-removable storage 350 including, but not limited to, magnetic or optical disks or tapes. Computing device 300 may also contain network connection(s) 380 that allow the device to communicate with other devices such as over the communication pathways described herein. The network connection(s) 380 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 300 may also have input device(s) 370 such as keyboards, keypads, switches, dials, mice, track balls, touch screens, voice recognizers, card readers, paper tape readers, or other well-known input devices. Output device(s) 360 such as printers, video monitors, liquid crystal displays (LCDs), touch screen displays, displays, speakers, etc. may also be included. The additional devices may be connected to the bus in order to facilitate communication of data among the components of the computing device 300. All these devices are well known in the art and need not be discussed at length here.

The processing unit 320 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 320 for execution. Example tangible, computer-readable media may include, but is not limited to, volatile media, non-volatile media, removable media and non-removable media implemented in any method or technology for storage of information such as computer readable instructions, data structures, program modules or other data. System memory 330, removable storage 340, and non-removable storage 350 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture 300 in order to store and execute the software components presented herein. It also should be appreciated that the computer architecture 200 may include other types of computing devices, including hand-held computers, embedded computer systems, personal digital assistants, and other types of computing devices known to those skilled in the art. It is also contemplated that the computer architecture 300 may not include all of the components shown in FIG. 30B, may include other components that are not explicitly shown in FIG. 30B, or may utilize an architecture different than that shown in FIG. 30B.

In an example implementation, the processing unit 320 may execute program code stored in the system memory 330. For example, the bus may carry data to the system memory 330, from which the processing unit 320 receives and executes instructions. The data received by the system memory 330 may optionally be stored on the removable storage 340 or the non-removable storage 350 before or after execution by the processing unit 320.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Moreover, the various components may be in communication via wireless and/or hardwire or other desirable and available communication means, systems and hardware. Moreover, various components and modules may be substituted with other modules or components that provide similar functions.

The computer architecture 300 includes software and/or hardware components and modules needed to enable the function of the modeling, simulation, and methods disclosed in the present disclosure. In some aspects, the computer architecture 300 may include artificial intelligence (A.I.) modules or algorithms and/or machine learning (M.L.) modules or algorithms (e.g., stored in the system memory 330, removable storage 340, non-removable storage 350, and/or a cloud database). The A.I. and/or M.L. modules/algorithms may improve the predictive power of the models, simulations, and/or methods disclosed in the present disclosure. For example, by using a deep learning, A.I., and/or M.L. model training including patient information and any relevant input data to the computational model, the predictive power of the computational model may be greatly enhanced. The A.I. and/or M.I. modules/algorithms also help improving sensitivity and specificity of the prediction as the database grows. In some aspects, the computer architecture 200 may include virtual reality (VR), augmented reality (AR) and/or mixed reality display(s), headset(s), glass(es), or any other suitable display device(s) as a part of the output device(s) 360 and/or the input device(s) 370. In some aspects, the display device(s) may be interactive to allow an user to select from options including with or without AR, with or without VR, or fused with real time clinical imaging to help clinician interact and make decisions.

Referring back to FIG. 29, at step 72, the image data 80 can include X-ray CT image data collected based on the patient 80. The image data 82 can be collected prior to a clinical procedure. For example, prior to performing a heart procedure (e.g., TAVR), the image data 82 can be generated based on the patient 80, which, as described herein, can be used to predict the outcome or risks associated with the clinical procedure. The image data 82 can characterize an anatomical region of the patient 80. The anatomical region can include one or more of the pulmonary root, pulmonary vein ostium, tricuspid annulus, superior vena cava, or inferior vena cava. The image data 82 can be stored in the memory 104 of the imaging device 104. In some examples, the image data 82 can be organized such as, for example, into systole data and/or diastole data. The image data 82 can be transmitted to the memory 204 of the image analysis device 200 such as, for example, via the network interface hardware 112 and the network interface hardware 206. Additionally, the image data 82 can be stored on or transmitted via an intermediary device that can include memory such as, for example, a cloud storage device or a portable memory.

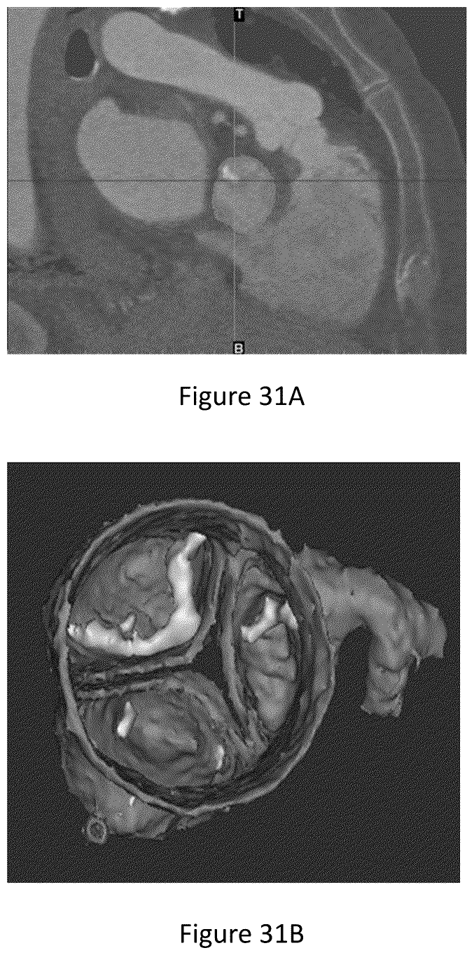

The image data 82 can be used to generate anatomical model data 84, such as shown in FIGS. 31A and 31B. The anatomical model data 84 can include computer-aided design (CAD) shapes composed of points, curves, surfaces, solids, or the like encoded into a machine readable format. In some examples, the image analysis device 200 can execute an image processing engine 210 provided on the memory 204. The image processing engine 210 can be programmed to generate the anatomical model data 84 based on the image data 82. The anatomical model data 84 can be provided in two-dimensions or three-dimensions. For example, CT images can include pixels or voxels indicative of relative intensity that can be encoded into a machine readable format such as, for example, Digital Imaging and Communications in Medicine (DICOM) format, X-ray, raw image data, or the like. Accordingly, the image processing engine 210 can include image processing methods that can evaluate CT images. Suitable commercial software toolkits including image processing methods are available such as, but not limited to, RadiAnt™, available from Medixant of Poznan, Poland, and Mimics available from Materialise of Leuven, Belgium.

In one embodiment, method for predictive heart valve simulation can include providing image data indicative of a heart of a patient. Anatomical regions of the heart can be segmented from the image data. The image data of the anatomical regions of the heart can be transformed, automatically with an image processing engine, into anatomical model data. The anatomical model data can include three-dimensional shapes of the anatomical regions of the heart. The anatomical model data can be transformed, automatically with a geometric modeling engine, into analytical model data. The analytical model data can include a three-dimensional mesh of the anatomical regions of the heart. The analytical model can be provided with a three-dimensional mesh of a surgical object. The analytical model data can be transformed, automatically with a numerical analysis engine, into a deformed analytical model. The deformed analytical model can be indicative of a deformed position of the anatomical regions of the heart and a deformed position of the surgical object.

In another embodiment, a method for predictive heart valve simulation, can include providing image data indicative of a heart of a patient. Anatomical regions of the heart can be segmented from the image data. The anatomical regions can include one or more calcific nodules, an aortic root having a coronary artery, and an aortic leaflet. The image data of the one or more calcific nodules, the aortic root, and the aortic leaflet can be transformed, automatically with an image processing engine, into anatomical model data. The anatomical model data can include three-dimensional shapes of the one or more calcific nodules, the aortic root, and the aortic leaflet. A deformed position of the aortic leaflet and the calcific nodule can be simulated automatically with one or more processors. A gap size can be quantified from the deformed position of the calcific nodule and the coronary artery of the aortic root.

In another embodiment, a method for predictive heart valve simulation can include providing image data indicative of a heart of a patient. Anatomical regions of the heart can be segmented from the image data. The anatomical regions can include one or more calcific nodules, an aortic root having a coronary artery, and an aortic leaflet. The image data of the one or more calcific nodules, the aortic root, and the aortic leaflet can be transformed, automatically with an image processing engine, into anatomical model data. The anatomical model data can include three-dimensional shapes of the one or more calcific nodules, the aortic root, and the aortic leaflet. The anatomical model data can be transformed, automatically with a geometric modeling engine, into analytical model data. The analytical model data can include three-dimensional meshes of the one or more calcific nodules, the aortic root, and the aortic leaflet. The analytical model data can be transformed, automatically with a numerical analysis engine, into a deformed analytical model. The deformed analytical model can be indicative of a deformed position of the calcific nodule and the coronary artery of the aortic root. A gap size can be measured from the deformed position of the calcific nodule and the coronary artery of the aortic root.

In a further embodiment, a method for predictive heart valve simulation can include providing image data indicative of a heart of a patient. The image data can include a calcific nodule, an aortic root having a coronary artery, and an aortic leaflet. Model parameters 90 can be measured from the anatomical model data. The model parameters 90 comprise a thickness t of the calcific nodule. A deformed position of the aortic leaflet and the calcific nodule can be determined, automatically with a parametric analysis engine, using the model parameters 90. The parametric analysis engine can model the aortic leaflet in a fully expanded position. A gap size can be quantified, automatically with the parametric analysis engine, from the deformed position of the calcific nodule and the coronary artery of the aortic root. The gap size can be a two-dimensional distance between a nodule point on the deformed position of the calcific nodule and an ostium point on the coronary artery of the aortic root.

At step 74, the image processing engine 210 can be further programmed to segment image data characterizing one or more anatomical regions from the image data 82. The imaging processing engine 210 can be programmed to generate the anatomical model data 84 based on the segmented image data. For example, the anatomical regions can include anatomy of the patient 80 that can be manipulated during a clinical procedure. In the example of TAVR, the anatomical regions can include an aortic root, native aortic leaflets, and calcific nodules. The calcific nodules can correspond to calcium based deposits that can develop within the patient 80. The calcific nodules can have an irregular geometric shape and can vary in size and shape for each patient 80. The segmentation can make use of various algorithms such as, for example, thresholding, edge detection, shape recognition, filtering, clustering, or the like. For example, the anatomical regions of CT images can include different ranges of intensity (e.g., pixel or voxel) relative to tissue stiffness. Once segmented, each of the anatomical regions can be transformed into a CAD shape within the anatomical model data 84.

At step 76, the analytical model data 86 can include mesh elements such as, for example, nodes and edges, that can be used for numerical analysis. In some examples, the image analysis device 200 can be configured to execute a geometric modeling engine 212 provided on the memory 204. The geometric modeling engine 212 can be programmed to generate analytical model data 86 based on the anatomical model data 84. Alternatively or additionally, the analytical model data 86 can include a surgical object representative of model implants, surgical instruments, or any other device that can interact with the anatomical regions of the patient 80 during the clinical procedure. For example, the surgical objects can include a three-dimensional model of a transcatheter aortic valve (TAV). Suitable commercial software toolkits for implementing the geometric modeling engine 212 can include, but not limited to, SolidWorks®.

In the example of TAVR, the analytical model data 86 can include meshes. The meshes can correspond to each of the aortic root, the aortic leaflets, the calcific nodules, and the surgical object. The meshes can be mapped to the CAD shapes of the anatomical regions and the surgical objects. For example, the nodes can be mapped to curves, surfaces, points, or the like of the anatomical model data 84. The nodes and edges of the mesh can be formed in a variety of shapes such as, for example, triangle, quadrilateral, tetrahedron, pyramid, hexahedron, or the like. In a test example, 10-node tetrahedral elements were mapped with a patch-independent algorithm to the anatomical regions corresponding to soft tissue regions. The stent was meshed using hexahedron elements. The total number of mesh elements varied for each patient, and was a function of the shape and size of the anatomical regions (e.g., aortic wall, aortic leaflets, coronary arteries, and calcific nodules).

At step 78, the image analysis device 200 can be configured to execute a numerical analysis engine 214 provided on the memory 204. The numerical analysis engine 214 can be programmed to map boundary conditions, and a system of equations to the analytical model data 86. The numerical analysis engine 214 can be programmed to solve the system of equations based on the boundary conditions to simulate the clinical procedure. For example, the numerical analysis engine 214 can be programmed for finite element analysis (FEA), computational fluid dynamics (CFD), deep learning simulation tools, or physics informed neural networks, reduced order models, or like. Suitable commercial software toolkits for implementing the numerical analysis engine 214 can include, but not limited to, ANSYS® available from ANSYS, Inc, PrecisionTAVI™ from DASI Simulations LLC, HeartGuide™ from FEOps/Materialise etc.

The numerical analysis engine 214 can be programmed to simulate the clinical procedure by assigning boundary conditions to the analytical model data 86 and manipulating the surgical objects to resemble the clinical procedure. In the example of TAVR, the numerical analysis engine 214 can be programmed to model an impact of the clinical procedure upon the anatomy of the patient 80. For example, the movement aortic leaflets and the calcific nodules can be modeled by the numerical analysis engine 204 to quantify an amount of coronary obstruction, paravalvular leakage, thrombosis, or a combination thereof. The material properties of the aortic root and the aortic leaflets can be considered to be linear elastic, and the calcific nodules and can be modeled by the numerical analysis engine 204 as rigid objects. The physical characteristics of the aortic root and the aortic leaflets can be mapped to the analytical model data 86, e.g., the mesh can be assigned a Young's modulus of about 2,000 kilopascal (kPa), Poisson's ratio of about 0.495, and a density of about 1,000 kilograms per meter squared (Kg/m³). Moreover, the surgical object can be modeled by the numerical analysis engine 204 as the stent, the TAV, the cylinder, or the like. For example, the surgical object can be modeled by the numerical analysis engine 204 as homogeneous isotropic stainless steel with a Young's modulus of about 205 gigapascal (GPa), Poisson ratio of about 0.275, and tensile strength of about 620 megapascals (MPa).

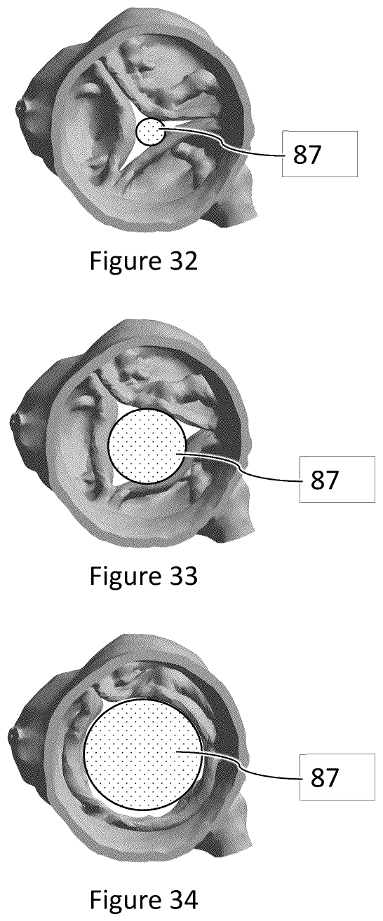

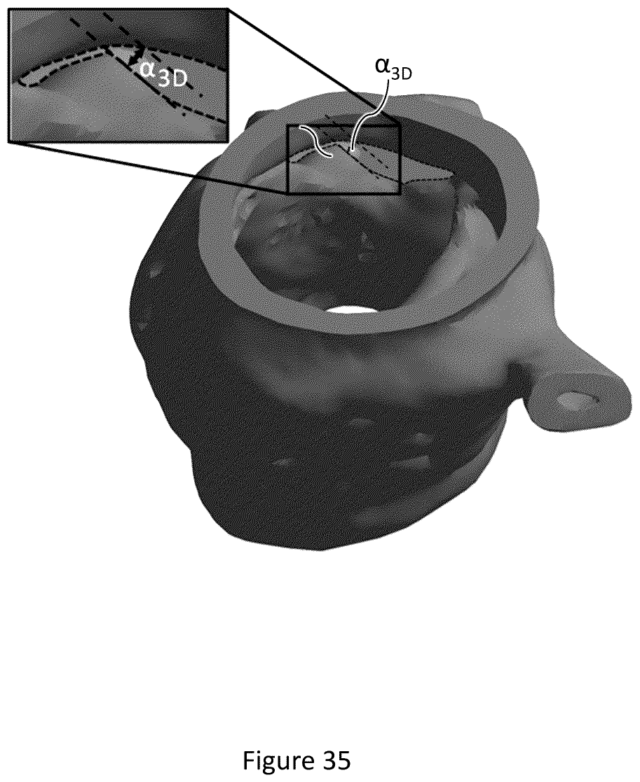

The numerical analysis engine 214 can be further programmed to generate a deformed analytical model 86 by modeling the impact of changing the dimensions of the surgical object. For example, as the dimensions of the surgical object change, the position of aortic leaflets and the calcific nodules can respond by changing position (e.g., the aortic leaflets can expand radially to cause the calcific nodules attached thereto to change position). Likewise, the surgical object can deform in response to interaction with the aortic leaflets and the calcific nodules. Accordingly, each deformed analytical model 86 can correspond to a deformed position of the aortic leaflets, the calcific nodules, the surgical object caused by the TAVR. Any number of deformed analytical models 86 can be defined to model an initial deployment of the surgical object, such as shown in FIG. 32, an intermediate deployment of the surgical object 87, such as shown in FIG. 33, a full deployment of the surgical object 87, such as shown in FIGS. 34 and 35, and any position there between.

In some examples, the surgical object 87 can be changed in the analytical model data 86 to generate additional deformed analytical models 88. Accordingly, the numerical analysis engine 214 can be programmed to predict the impact of the use of different sizes or types of TAV's upon the anatomy of the patient 80. Moreover, the surgical object 87 can be repositioned in the analytical model data 86 to determine the impact of changes in positioning upon the deformed analytical models 88. For example, the TAV can have an insertion depth (e.g., a distance between the top portion of the TAV and the annulus of the aortic root). Additionally, a pitch angle and yaw angle relative to a centerline of the aortic root can be modeled by the numerical analysis engine 214. Accordingly, the pitch angle, yaw angle, insertion depth, or a combination thereof, can be modeled to quantify a sensitivity of the patient 80 to the TAVR. In some examples, deformed analytical models 86 can be generated consecutively, or in parallel, to allow for direct comparison of different sizes, types, or positions of TAV's. For example, each of the deformed analytical models 86 can be depicted on the display 208. Thus, an efficacy of each TAV can be visualized prior to the clinical procedure, for example, prior to performing TAVR. The numerical analysis engine 214 can further be programmed to simulate blood flow properties for any of the deformed analytical models 88.

Referring collectively to FIGS. 34 and 35, after modeling the deformation of the aortic leaflets caused by full deployment of the surgical object a gap size can be determined. The gap size can correspond to the shortest three-dimensional distance between a coronary ostium of the coronary artery and a potential obstruction such as, for example, a calcific nodule on the aortic leaflets, or the aortic leaflets. In other words, the gap size can be determined using the position of the aortic leaflets after TAV stent deployment. Generally, the gap size can be correlated to risk of coronary obstruction. Specifically, for proper heart function blood must travel over the aortic leaflets and reach the coronary ostium. During TAV stent deployment, aortic leaflets can be forced towards the coronary arteries to accommodate the new valve prosthesis. A life-threatening complication known as coronary ostium obstruction can occur when the aortic leaflets are forced into a position that blocks the coronary ostia, cutting off blood flow to the rest of the heart.

Accordingly, a small gap size (e.g., less than about 3 mm) can indicate that the coronary artery is blocked. Moreover, it is noted that for some individuals, the gap size can be measured relatively easily. However, for other individuals, especially those with high risk of coronary obstruction, additional views and inspection may be required to determine the gap size. Accordingly, the deformed analytical model 82 can improve the accuracy of the diagnosis by providing a full three-dimensional geometric representation of the calcific nodule, the aortic leaflets, and the coronary artery.

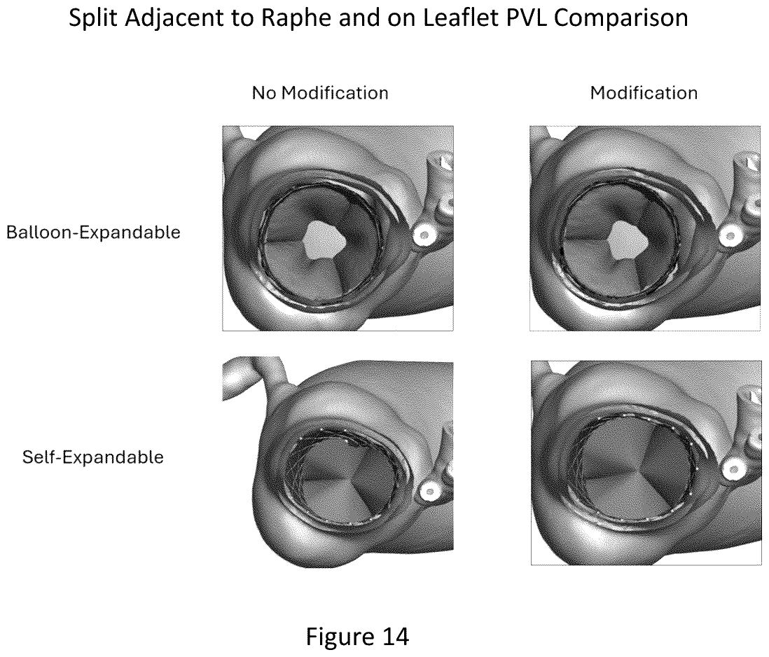

Referring collectively to FIGS. 14, 34, and 35, after modeling the deformation of the aortic leaflets caused by full deployment of the surgical object a gap size can be determined to quantify para-valvular leakage, i.e., undesired blood flow between the TAV and the annulus of the aortic root. The gap size can correspond to the largest three-dimensional distance between the stent or artificial leaflets and the annulus of the aortic root. Generally, the gap size can be correlated to risk of para-valvular leakage.

Referring collectively to FIGS. 16, 30, and 36, the numerical analysis engine 214 can simulate blood flow properties for any of the deformed analytical models 82. For example, the geometry of the deformed analytical model 82 corresponding to full deployment of the surgical object can be used for CFD (or any manner of reduced order fluids simulation) to model blood flow properties in aortic root region under different conditions. In some embodiments, the blood flow can be used to quantify para-valvular leakage. For example, the amount and rate of blood flow flowing between the TAV and the aortic root can be indicative of the relative risk of para-valvular leakage. Alternatively or additionally, the blood flow properties of the deformed analytical model can quantify thrombosis. Thrombosis can correspond to localized coagulation or clogging of the blood induced by TAVR. Accordingly, the blood flow can identify TAVR induced regions of blood stasis. Additionally, the CFD can be used to model a flow pattern of contrast agent flow in coronary artery, which can be used to validate the numerical analysis engine 214 or the efficacy of the modeled surgical procedure with data collected after the surgical procedure is performed. For example, comparing the arrangement of the calcific nodule arrangement and flow patterns in the CFD against aortographic images captured after the surgical procedure can provide insight into the accuracy of deformed analytical model 82 and the CFD/reduced order fluids simulations.

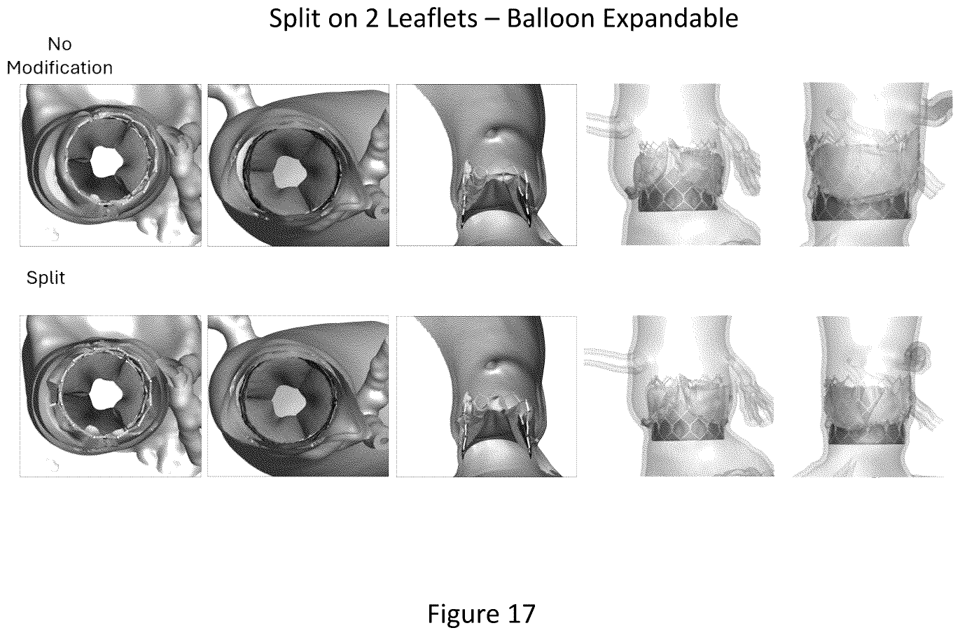

Referring collectively to FIGS. 17-23, 30A, 30B, 31A, and 31B, step 78 (shown in FIG. 29) can include a process for generating parameters indicative of the anatomical regions of the patient 80. In some embodiments, parameters can be generated directly or indirectly from the image data 82. For example, the image data 82 can include a plurality of slices of CT data of the left coronary leaflet and the right coronary leaflet, which can be directly measured to determine parameters of the anatomical regions such as, for example, coronary ostium height from the annulus baseline, annulus diameter, and sinotubular junction (STJ) diameter, which can correspond to the final position of the coronary leaflets after deployment, can be obtained from the slices of CT data of the left coronary leaflet and the right coronary leaflet. Alternatively or additionally, model parameters 90 can be determined from the plurality of slices of CT data of the left coronary leaflet and the right coronary leaflet. The model parameters 90 can include height of coronary artery from annulus, thickness of the calcific nodule on the left coronary leaflet, thickness of the calcific nodule on the right coronary leaflet, a projection of coronary ostium diameter on the annulus to STJ line, sinus width between coronary ostium and the annulus to STJ, leaflet length of the left coronary leaflet, and leaflet length of the right coronary leaflet. Since the aortic leaflets undergo the most strain during diastole, the image data 82 can be captured in the diastolic phase of the cardiac cycle. In further embodiments, the parameters, the model parameters 90, or both can be collected from the anatomical model data 84.

Referring collectively to FIGS. 17, 24, 25, and 30 the step 78 (as shown in FIG. 29) can include a process for simulating a surgical procedure. In some embodiments, the image analysis device 200 can automatically execute a parametric analysis engine 216 provided on memory 204. The parametric analysis engine 216 can be configured to simulate the impact of a surgical procedure upon the size and the location of the calcium nodule using the model parameters. When the parametric analysis engine 216 simulates TAVR, a gap size can be determined by modeling the coronary leaflets in a fully expanded position (FIGS. 23 and 35) due to TAV stent deployment. The gap size can correspond to a two-dimensional or three-dimensional distance between the tip of the coronary leaflet and coronary ostium of the coronary artery. Generally, the gap size can be correlated to risk of coronary obstruction. It is noted that the parametric analysis engine 216 can model anatomy of the patient 80 in two-dimensions to determine the gap size.

The parametric analysis engine 216 can determine the location of two points: nodule point Pc, which can correspond to the position of the calcific nodule of the aortic leaflet; and ostium point Po, which can correspond to the position of the upper edge of the coronary ostium of the coronary artery. Accordingly, the gap size α_{2D} can be calculated by the parametric analysis engine 216 using Equation 1.

$${α}_{2 D} = \sqrt{{( Δ x )}^{2} + {( Δ y )}^{2}} , \qquad ( Equation l )$$

where Δx is a horizontal offset (x-direction) between the nodule point Pc and the ostium point Po, and Δy is a vertical offset (y-direction) between the nodule point between Pc and the ostium point Po. The horizontal offset Δx can be calculated using Equation 2 and the vertical offset Ay can be calculated using Equation 3.

$$Δ x = w - t \qquad ( Equation 2 )$$

$$Δ y = h + d - l \qquad ( Equation 3 )$$

where the following model parameters are used: the sinus width wat the ostium level of the coronary artery, the thickness t of the calcific nodule on the tip of the aortic leaflet, the leaflet length l, height h of the coronary ostium of the coronary artery, and coronary ostium diameter d of the coronary artery. The parametric analysis engine 216 can calculate the gap size α_{2D} for both left and right coronary ostium of the coronary arteries.

Referring to FIG. 21 anatomical model data was reconstructed for a given patient. Aortic and ventricular views of the anatomical model data for each patient's reconstructed aortic root geometry are provided. Calcific nodules (colored yellow) were reconstructed separately from the aortic root (colored pink) and then added to the leaflets. The geometry for Patient, who has a tri-leaflet calcified aortic valve is shown. Although basic characteristics of all the patients such as tri-leaflet valves, two coronary arteries, and arrangement of the cusps are similar, each patient has a unique aortic geometry with different patterns and severity of calcification (i.e., different size, shape, and position of calcific nodules).

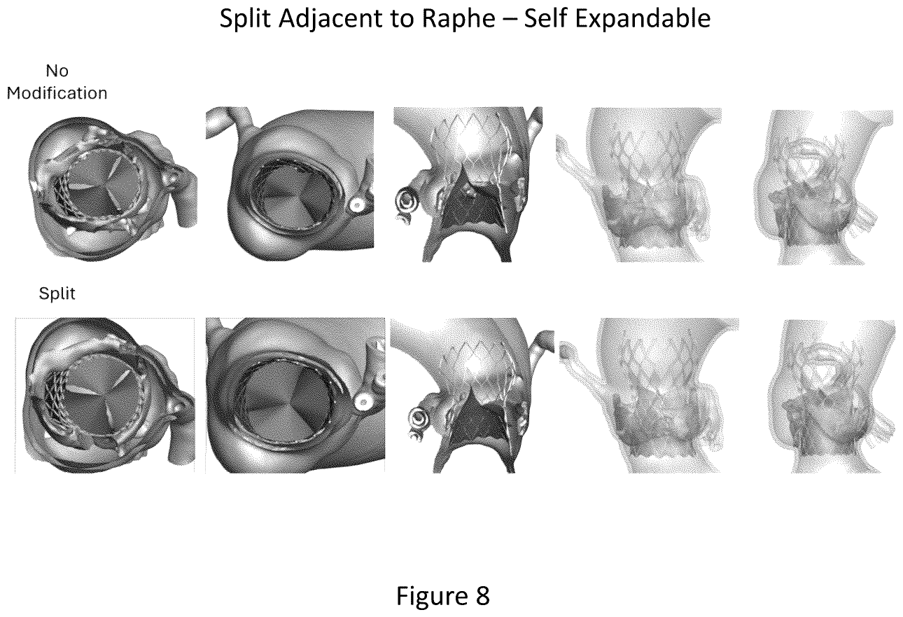

Referring collectively to FIGS. 8 and 17, analytical model data was generated from the anatomical model data. The numerical analysis engine was used to determine deformed analytical models from the analytical model data. The deformed analytical models corresponding to TAV stent deployment were extracted. Cross-sectional views of both left and right coronary arteries were selected from the three-dimensional geometry to show the final position of leaflets relative to the left and right coronary ostia. These cross-sections include the ostium centerline as well as maximum calcification thickness on the leaflet tip. Cross-sectional views of simulated post-deployment anatomy of the nine patients for both left and right coronary ostium are provided. For ease of recognition, the edge of the leaflets are highlighted in green or blue, and calcific nodules on the leaflets are highlighted in yellow. For patients previously determined the parametric analysis engine as being high risk for coronary obstruction, the three-dimensional cross-sectional views also illustrate the possibility of the native leaflets blocking the ostia.

It is noted that the terms “substantially” and “about” can be used herein to represent the inherent degree of uncertainty that can be attributed to any quantitative comparison, value, measurement, or other representation. These terms are also used herein to represent the degree by which a quantitative representation can vary from a stated reference without resulting in a change in the basic function of the subject matter at issue.

While particular embodiments have been illustrated and described herein, it should be understood that various other changes and modifications can be made without departing from the spirit and scope of the claimed subject matter. Moreover, although various aspects of the claimed subject matter have been described herein, such aspects need not be used in combination. It is therefore intended that the appended claims cover all such changes and modifications that are within the scope of the claimed subject matter.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 508. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in either one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method comprising:
obtaining data related to a heart medical procedure for a patient, the data comprising patient-specific data comprising 3D computational analysis generated from pre-procedural medical imaging, device data related to at least one device to be deployed in the heart medical procedure, and interactive input from medical personnel;
generating, based on the data, options of modification strategies comprising device modifications to the at least one device and anatomical modifications to the patient's anatomy;
simulating the heart medical procedure and generating a simulation outcome for each option of the modification strategies;
selecting a modification strategy that corresponds to the simulation outcome having a desired heart medical procedure result; and
generating procedural recommendations comprising device deployment instructions and modification parameters to guide the heart medical procedure in real life.

2. The computer-implemented method of claim 1, wherein the at least one device comprises one or more of: a heart valve, a guidewire, a cutting device, an electrosurgical splitter, a catheter, a deployment system of the at least one device, and a left atrial appendage occlusion (LAAO) device.

3. The computer-implemented method of claim 1, wherein the data further comprises data communicated in real-time during the heart medical procedure from a sensor system comprising one or more sensors incorporated in the at least one device or a deployment system of the at least one device.

4. The computer-implemented method of claim 1 is performed in real-time during the heart medical procedure.

5. The computer-implemented method of claim 1 further comprising generating a selection of at least one modification device for the selected modification strategy, wherein the at least one modification device comprises a cutting device, a splitter, or a combination thereof.

6. The computer-implemented method of claim 5, wherein the at least one modification device comprises a cutting device for heart valve implementation.

7. The computer-implemented method of claim 1 further comprising displaying the procedural recommendations via a visual display capable of allowing the medical personnel to provide the interactive input.

8. The computer-implemented method of claim 1, wherein generating the simulation outcome for each option of the modification strategies comprises using quantitative risk metrics to evaluate outcomes of the modification strategies.

9. The computer-implemented method of claim 1, wherein generating the simulation outcome for each option of the modification strategies comprises ranking the modification strategies with quantitative justification and valve performance for each modification strategy.

10. The computer-implemented method of claim 1, wherein the heart medical procedure comprises a transcatheter aortic valve replacement (TAVR), a valve-in-valve (ViV) replacement, or a surgical valve replacement.

11. The computer-implemented method of claim 1, wherein the patient-specific data comprises at least one of: native valve morphology, physicologic conditions, patient demographics, structural measurements of aortic root, annulus, sinotubular junction, coronary heights, leaflet calcification volume and distribution dynamic physiologic parameters, and the pre-procedural medical imaging data from CT, MRI, echocardiography (TTE or TEE), fluoroscopy, or catheter-derived hemodynamic recordings.

12. The computer-implemented method of claim 1, wherein simulating the heart medical procedure comprises performing a predictive simulation using one or more computational models selected from: nonlinear finite element analysis (FEA), fluid-structure interaction (FSI) modeling, computational fluid dynamics (CFD), reduced-order surrogate models, simplified force-driven particle models, and machine-learning-based risk estimation models.

13. The computer-implemented method of claim 1, wherein the anatomical modifications comprising one or more of: splitting, releasing, incising, thinning, scoring, and/or debulking to alternate native leaflets, commissures, raphe tissue, or calcified regions.

14. The computer-implemented method of claim 1, wherein the device modifications comprise one or more of: structural alterations to the at least one device, size or dimensional adjustments of the at least one device, orientational adjustments of the at least one device, alignment adjustments of the at least device, balloon-related modifications for a balloon-expandable implant device, adding sealing elements to a leaflet and/or a skirt of the at least one device.

15. A system comprising:
a memory comprising instructions; and
a processor configured to execute the instructions which, when executed, cause the processor to:
obtain data related to a heart medical procedure for a patient, the data comprising patient-specific data comprising 3D computational analysis generated from pre-procedural medical imaging, device data related to at least one device to be deployed in the heart medical procedure, and interactive input from medical personnel;
generate, based on the data, options of modification strategies comprising device modifications to the at least one device and anatomical modifications to the patient's anatomy;
simulate the heart medical procedure and generate a simulation outcome for each option of the modification strategies;
select a modification strategy that corresponds to the simulation outcome having a desired heart medical procedure result; and
generate procedural recommendations comprising device deployment instructions and modification parameters to guide the heart medical procedure in real life.

16. The system of claim 15, wherein the at least one device comprises one or more of: a heart valve, a guidewire, a cutting device, an electrosurgical splitter, a catheter, a deployment system of the at least one device, and a left atrial appendage occlusion (LAAO) device.

17. The system of claim 15, wherein the data further comprises data communicated in real-time during the heart medical procedure from a sensor system comprising one or more sensors incorporated in the at least one device or a deployment system of the at least one device.

18. The system of claim 15, wherein the anatomical modifications comprise one or more of: splitting, releasing, incising, thinning, scoring, and/or debulking to alternate native leaflets, commissures, raphe tissue, or calcified regions.

19. The system of claim 15, wherein the device modifications comprise one or more of: structural alterations to the at least one device, size or dimensional adjustments of the at least one device, orientational adjustments of the at least one device, alignment adjustments of the at least device, balloon-related modifications for a balloon-expandable implant device, adding sealing elements to a leaflet and/or a skirt of the at least one device.

20. A non-transitory machine-readable storage medium comprising machine-readable instructions for causing a processor to execute a method, the method comprising:
obtaining data related to a heart medical procedure for a patient, the data comprising patient-specific data comprising 3D computational analysis generated from pre-procedural medical imaging, device data related to at least one device to be deployed in the heart medical procedure, and interactive input from medical personnel;
generating, based on the data, options of modification strategies comprising device modifications to the at least one device and anatomical modifications to the patient's anatomy;
simulating the heart medical procedure and generating a simulation outcome for each option of the modification strategies;
selecting a modification strategy that corresponds to the simulation outcome having a desired heart medical procedure result; and
generating procedural recommendations comprising device deployment instructions and modification parameters to guide the heart medical procedure in real life.
